(19) United States
(12) Patent Application Publication
(10) Pub. No.: US 2026/0144482 A1
(43) Pub. Date: May 28, 2026

(54) Clinician-Bounded Closed-Loop Control Systems with Governed Autonomy for Therapeutic Medical Devices

(71) Applicant: OneSource Solutions International, Inc., Sudbury, MA (US)

(72) Inventors: Harold Arkoff, Sudbury, MA (US); Vedran Jukic, Trieste (IT)

(73) Assignee: OneSource Solutions International, Inc., Sudbury, MA (US)

(21) Appl. No.: 19/454,818

(22) Filed: Jan. 21, 2026

Related U.S. Application Data
(63) Continuation-in-part of application No. 19/270,319, filed on Jul. 15, 2025, now Pat. No. 12,562,280.
(60) Provisional application No. 63/672,848, filed on Jul. 18, 2024.

(51) Int. Cl. A61B 5/00

(52) U.S. Cl. CPC A61B 5/4836; A61B 5/0002; A61B 5/7275; A61B 2560/0228

(57) ABSTRACT

A clinician-bounded closed-loop control system for therapeutic medical devices in regulated clinical environments is disclosed. The system receives physiologic monitoring signals and therapeutic-device telemetry and computes a patient state representation including confidence indicators. The patient state is evaluated relative to clinician-authored order sets defining autonomy levels and multi-dimensional physiologic and device corridors. When permitted, the system generates candidate therapeutic-device control commands and validates admissibility using device-specific safety grammars enforcing semantic bounds and transition constraints. Candidate commands are classified by risk, with high-risk actions gated on clinician authorization. Authorized commands are dispatched via a validation and execution interface that receives acknowledgements and post-dispatch telemetry. Conformance is evaluated using persistence criteria to distinguish transient excursions from sustained deviation. Upon sustained deviation or execution failure, governed corrective actions are initiated. Governance enforcement provides auditability through execution authorization artifacts and ledger-gated completion while preserving clinician authority and regulatory accountability.

### CROSS-REFERENCE TO RELATED APPLICATIONS

This application is a Continuation in Part of U.S. patent application Ser. No. 19/270,319, filed Jul. 15, 2025, entitled “Multilingual Healthcare System with Personalized Medical Assistant, Decision Support, and Closed Loop Device Control,” the disclosure of which is incorporated by reference in its entirety.

This application also claims the benefit of U.S. Provisional Patent Application No. 63/672,848, filed Jul. 18, 2024, the disclosure of which is incorporated by reference in its entirety.

In the event of any inconsistency between an incorporated reference and this specification, the disclosure of this specification controls.

### FIELD OF THE INVENTION

The present invention relates to computerized control systems for therapeutic medical devices operating in regulated clinical environments. More particularly, the invention relates to clinician-bounded, policy-governed closed-loop control systems that generate, validate, authorize, and execute therapeutic-device control actions in response to physiologic monitoring data and device telemetry, subject to enforceable safety constraints, authorization requirements, and auditable governance mechanisms.

In exemplary embodiments, the invention provides technical mechanisms for executing therapeutic-device control actions only when such actions satisfy clinician-defined intent, physiologic safety constraints, and governance conditions, including traceable authorization, tamper-evident recordation, and execution-path enforcement. The invention further relates to systems that support rollback, safe-mode transition, suspension of actuation, and other corrective procedures when monitored outcomes deviate from expected or permitted behavior.

### BACKGROUND OF THE INVENTION

Modern clinical environments produce continuous streams of physiologic monitoring signals and therapeutic-device telemetry from bedside monitors, infusion pumps, ventilators, hemodynamic devices, and related systems. Despite the availability of such data, safe and effective patient management continues to rely on clinician judgment, institutional policy, and regulatory accountability. Therapeutic-device control in regulated care settings must therefore balance responsiveness to real-time patient conditions with enforceable safety limits, clinician oversight, and auditable traceability.

Existing computerized systems generally fall into two categories. Some systems provide passive clinical decision support, generating recommendations, alerts, or visualizations that require manual clinician action and that lack technical mechanisms to ensure execution, confirmation, or accountability. Other systems attempt automated or closed-loop control of therapeutic devices but operate with limited governance, insufficient confirmation behavior, or inadequate mechanisms for enforcing clinician-bounded authority, particularly in environments subject to regulatory oversight.

practical clinical “autopilot,” as contemplated herein, is not an unsupervised autonomous clinician and does not replace professional judgment. Rather, there is a need for a clinician-bounded control system that continuously receives physiologic and device telemetry, interprets such data to produce governed findings accompanied by confidence or signal-quality indicators, and acts only within permissions, bounds, and protocols explicitly defined by a clinician-authored order set and applicable institutional policy. When evidence is insufficient, signals conflict, or uncertainty exceeds acceptable thresholds, such a system should suppress autonomous actuation and instead request additional measurements, increased monitoring, or clinician confirmation, while reporting its findings in a form suitable for clinical decision-making.

Clinical management further frequently involves titration-to-effect, in which therapy is adjusted incrementally, patient response is observed over time, and subsequent adjustments are made based on that observed response. Safe titration requires time-based persistence and confirmation logic to distinguish transient artifacts or short-lived excursions from sustained conditions warranting intervention. In this context, measurement cadence itself may function as a controllable parameter, such as temporarily increasing non-invasive blood pressure measurement frequency during periods of instability and relaxing the cadence as stability is demonstrated.

Conventional systems often lack unified mechanisms for enforcing such behavior at runtime. In particular, many systems fail to technically bind therapeutic-device actuation to clinician-defined intent, authorization requirements, and auditability, instead relying on retrospective logging or procedural safeguards. Additionally, omission events—such as failure to reassess, confirm, or escalate within a required time window—are frequently under-managed, despite posing risks comparable to incorrect actions.

Accordingly, there is a need for systems that combine continuous interpretation of physiologic and device telemetry with clinician-defined corridor constraints, bounded execution of therapeutic-device control actions, time-based persistence and confirmation logic, secure execution with acknowledgements and corrective procedures, and governance enforcement mechanisms that render proposed and executed actions explainable, authorized when required, and traceable for clinical, regulatory, and legal accountability.

### SUMMARY OF THE INVENTION

The present invention provides a policy-governed clinical control system for use in regulated healthcare environments that issues and enforces therapeutic-device control commands affecting physical operation of one or more therapeutic devices, while maintaining clinician-bounded authority, safety constraints, and auditable accountability. In exemplary embodiments, the invention operates as a runtime machine-control enforcement system that sits directly in the actuation path of therapeutic devices and technically prevents unsafe or unauthorized physical device operation, rather than merely providing decision support, recommendations, or post hoc analysis. The system enables clinician-bounded, closed-loop titration-to-effect of therapeutic devices under an active clinician-authored order set and enforceable governance controls. In exemplary embodiments, a clinician specifies therapeutic intent by defining goals, bounds, and permissions using multi-dimensional corridor constraints and an autonomy level, which together delineate a permitted action space, applicable step-size and rate-of-change limits, and whether particular classes of actions require authorization evidence prior to execution.

In operation, the system receives real-time physiologic monitoring signals and therapeutic-device telemetry and computes a patient state representation comprising physiologic state variables, temporal information, and signal-quality or confidence indicators. The system processes incoming data to produce governed findings that control whether therapeutic-device control commands may be proposed or executed, and to assess whether available evidence is sufficient to support autonomous actuation. When evidence is insufficient, conflicting, or degraded, the system suppresses autonomous actuation and instead generates governed confirmation or tool requests, such as requests for additional measurements, increased monitoring cadence, or confirmatory data, while reporting the findings, associated uncertainty, and recommended next steps to an authorized clinician interface.

Within the clinician-defined corridor constraints and autonomy level, the system generates candidate therapeutic-device control commands based on the patient state representation and validates admissibility using device-specific safety constraints. Such constraints may include semantic bounds on parameter values and transition constraints limiting step size or rate of change between successive device settings. When a candidate control command is classified as high-risk according to the active order set or applicable policy thresholds, the system gates execution on receipt of authorization evidence from an authorized clinician. The system further applies time-based persistence criteria so that control actions and deviation responses are triggered only when nonconforming conditions persist over defined durations or sample windows, thereby reducing inappropriate responses to transient artifacts, noise, or momentary threshold crossings.

Following dispatch of an admissible and permitted therapeutic-device control command, the system monitors post-dispatch physiologic and device telemetry over a defined observation window and performs bounded titration-to-effect response updating based on observed post-dispatch telemetry. Using this response information, the system determines whether the patient state is converging toward desired corridor bounds, overshooting, oscillating, or failing to respond as expected. When sustained deviation conditions, instability patterns, or policy-defined triggers are detected, the system selects and initiates appropriate corrective procedures, including rollback to a prior known-safe configuration, compensatory adjustments, transition of the therapeutic device to a predefined safe mode, or suspension of further autonomous actuation pending reauthorization. In certain embodiments, the system further detects omission events, such as failure to complete required confirmations or reassessments within specified time windows, and treats such omissions as governed events subject to corrective action.

In some embodiments, governance is enforced directly within the execution path through ledger-gated completion and execution authorization artifacts bound to policy identifiers, corridor definitions, and order set versions. In exemplary embodiments, governed records and execution authorization artifacts are not merely audit logs, but constitute runtime gating prerequisites such that progression of the closed-loop system to subsequent actions is prevented unless required compliance evidence is successfully generated and validated. In such embodiments, dispatch, acknowledgement, and/or completion of therapeutic-device control actions are treated as valid only when compliant governed records are successfully created and appended to a tamper-evident record store. If such a compliant governed record is not created, the system prevents dispatch or treats the attempted action as failed, thereby inhibiting further autonomous progression of the closed-loop control system. This execution-path enforcement provides technical assurance that therapeutic-device control actions are explainable, authorized when required, and traceable, thereby supporting clinical safety, regulatory compliance, and post hoc accountability in regulated care settings.

In exemplary embodiments, the invention is a runtime machine-control governance system that sits in the actuation path for medical devices and prevents unsafe or unauthorized therapeutic-device commands from executing by enforcing clinician-authored boundaries, safety constraints, and required authorizations at runtime. The system further monitors post-dispatch conformance and initiates corrective procedures when sustained deviation is detected, while producing an auditable record binding evidence, constraints, authorizations, device actions, and outcomes.

### BRIEF DESCRIPTION OF THE DRAWINGS

FIG. 1 is a block diagram illustrating a clinician-bounded, policy-governed therapeutic-device control system with execution-path governance and ledger-gated closed-loop progression.

FIG. 2 is a block diagram illustrating a governed, tamper-evident record store that maintains cryptographically linked execution records used to enforce authorization, auditability, and completion gating.

FIG. 3 is a block diagram illustrating a clinician-bounded, policy-governed therapeutic-device control loop implementing physiologic corridor enforcement and autonomy-limited actuation.

FIG. 4 is a block diagram illustrating a constraint and safety grammar validator that evaluates admissibility of therapeutic-device control commands using semantic bounds and coupled transition constraints.

FIG. 5 is a block diagram illustrating a validation and execution interface that performs authorization validation, command dispatch, telemetry feedback processing, and system-initiated rollback or safe-mode control.

FIG. 6 is a block diagram illustrating an optional governed deployment and closed-loop surveillance controller that supports system-level governance enforcement, omission detection, and autonomy state management.

### DETAILED DESCRIPTION OF THE INVENTION

### Overview of the Clinician-Bounded Autopilot Assistant

In regulated clinical environments, a practical “autopilot” is not an unsupervised or independently autonomous clinician. As used herein, the term “autopilot assistant” refers to a clinician-bounded clinical control system that operates under explicit governance, authorization, and safety constraints. The autopilot assistant is configured to continuously receive authorized physiologic monitoring telemetry and therapeutic-device telemetry, to interpret that telemetry into governed findings that include associated confidence indicators, and to propose or execute therapeutic-device control actions only within the permissions, bounds, and conditions defined by an active clinician-authored order set and applicable institutional policy.

The autopilot assistant does not originate therapeutic intent or clinical objectives. Instead, it functions as a delegated execution system that performs bounded titration-to-effect strictly within clinician-defined corridors. In exemplary embodiments, the clinician-authored order set defines which therapies or devices may be controlled, the permissible ranges for physiologic variables and device parameters, the allowable magnitude and rate of parameter transitions, the required measurements or confirmations that must precede action, and the conditions under which escalation or additional authorization is required. The system enforces these constraints at runtime such that actions outside the defined authority are neither proposed nor executed.

During operation, the autopilot assistant continuously forms governed findings by distinguishing observed telemetry from inferred patient or device state and by associating each finding with one or more confidence or signal-quality indicators. When uncertainty is elevated, telemetry sources conflict, response behavior fails to converge, or a candidate action is classified as high-risk, the system does not proceed with actuation. Instead, the system initiates a governed escalation in which it generates a clinician-facing communication that describes the observed evidence, the system's interpretation and associated uncertainty, the actions attempted within permitted bounds, and the specific confirmation, authorization, or order revision required before further actuation may occur.

In certain embodiments, the clinician-bounded autopilot assistant is architected to reflect established critical-care delegation patterns in which a physician defines therapeutic intent and boundaries while a delegated bedside role performs continuous observation, bounded adjustment, and escalation. Technically, this is implemented by treating clinician inputs—such as modifying corridor bounds, revising an order set, responding to a confirmation request, issuing an override, or authorizing a high-risk action—as governed control inputs that update the system's permitted action space. As a result, clinician dialogue and order modification are integrated into the closed-loop control process rather than treated as external interruptions.

The system operates as a repeating closed-loop process in which telemetry is acquired and interpreted, a governed patient state representation is maintained with associated confidence indicators, evidence sufficiency is evaluated prior to any action, and candidate actions are generated only when permitted by the active order set. When evidence is sufficient and the proposed action is within permitted bounds and risk classification, the system may execute the action under governance control. When evidence is insufficient or risk is elevated, the system reports findings, uncertainty, and recommendations to an authorized clinician interface and awaits further instruction.

In exemplary embodiments, the autopilot assistant operates under an explicitly configured autonomy level associated with the active order set. The autonomy level defines whether the system may operate in a report-only posture, a recommendation posture, or a bounded actuation posture, and whether certain classes of actions require explicit authorization evidence prior to execution. The system is further configured to dynamically downgrade its autonomy level when evidence sufficiency criteria are not met, when telemetry quality degrades, when acknowledgments are ambiguous, or when governance dependencies fail, thereby preventing unsafe or unauthorized actuation.

When evidence is insufficient, when telemetry sources conflict, or when signal quality is degraded, the system suppresses actuation and generates a governed confirmation or tool request. Such requests may include repeating a measurement, temporarily increasing monitoring cadence for confirmation, obtaining a confirmatory laboratory or point-of-care value, verifying sensor placement or calibration, or requesting clinician input. In exemplary implementations, the system provides an explanation identifying why the additional information is required and how it affects subsequent decision-making within the closed loop.

In some embodiments, the system escalates when a deviation persists despite bounded actions, when non-convergence or oscillatory behavior is detected, or when a candidate action is classified as high-risk under the active order set or policy. Escalation may include requiring authorization evidence prior to execution, recommending clinician review of competing objectives, downgrading autonomy, or suspending further actuation pending reauthorization or revision of the active order set.

In certain embodiments, the autopilot assistant supports titration-to-effect by learning patient-specific response characteristics from observed outcomes of executed actions. The system may maintain a patient-specific response profile that informs selection among otherwise permissible low-risk adjustments or triggers earlier escalation when response behavior deviates from expectations. All updates to such patient-specific profiles are performed under governance control and are recorded in association with applicable order set versions, policy identifiers, and confidence indicators to preserve auditability and accountability.

Accordingly, the embodiments described herein provide a clinician-bounded autopilot assistant that listens to monitored data, interprets patient and device state under governance control, proposes or executes only those actions that are explicitly authorized within clinician-defined corridors, requests confirmation when evidence is insufficient, escalates when uncertainty or risk is elevated, and preserves traceability and auditability suitable for regulated clinical environments.

### System Architecture and Core Modules

This section describes an optional embodiment of the canonical governed closed-loop operation, corresponding primarily to the Validate/Admissibility stage, and is not required in all implementations.

The interface may ingest telemetry from bedside monitors, wearable sensors, invasive monitoring systems, and therapeutic devices, as well as clinician-entered values or structured data obtained from electronic medical record systems or middleware platforms. In exemplary implementations, the interface normalizes incoming data into a governed internal representation and applies validation checks to detect missing values, stale measurements, or degraded signal quality before downstream processing.

In some embodiments, the system distinguishes between actuatable therapeutic devices and non-actuatable clinical data sources. Actuatable therapeutic devices are those that expose programmable control parameters and are capable of receiving external commands that affect therapy delivery or device operation. Non-actuatable sources include monitoring systems, laboratory results, imaging systems, and other observational data sources that do not accept control commands. For non-actuatable sources, the system operates in a report-first posture, generating governed findings and escalation recommendations without attempting actuation. For actuatable devices, the system restricts command dispatch to action classes for which a device-specific safety grammar and admissibility validation are available and permitted by the active order set and autonomy level. By way of non-limiting example, a device-specific safety grammar may include rules specifying that an infusion rate adjustment shall not exceed a defined step-size increment per control iteration, that cumulative rate changes shall not exceed a maximum rate-of-change over a defined time window, and that certain parameter transitions are prohibited unless prerequisite device states or coupled parameter conditions are satisfied.

The therapeutic-device interface is configured to communicate with one or more actuatable therapeutic devices associated with the patient. This interface provides secure command transport, device-specific command formatting, and a feedback channel through which the system receives acknowledgements, negative acknowledgements, error codes, or completion signals from the device. By receiving explicit device responses, the system can determine whether a dispatched command was accepted, rejected, executed, or left in an ambiguous state, and can apply governance rules and safe behaviors accordingly.

state estimation module processes the physiologic monitoring signals and therapeutic-device telemetry to generate a patient state representation. The patient state representation represents the current and recent physiologic and device state of the patient and may include physiologic variables, trend features derived over time windows, device status indicators, and at least one confidence or signal-quality indicator associated with one or more inputs. In some embodiments, the state estimation module performs artifact detection, reconciles multiple sources providing overlapping measurements, and applies confidence-weighted fusion to generate a governed representation suitable for downstream decision-making.

The system includes an order-set and corridor module that obtains clinician intent from an active clinician-authored order set and represents that intent as multi-dimensional corridor constraints and associated governance parameters. The order set defines permitted physiologic targets and bounds, allowable therapeutic-device parameter ranges, permissible action classes, autonomy level settings, and high-risk criteria that determine when authorization evidence is required prior to execution. By representing clinician intent as corridors and governance rules rather than as fixed commands, the system supports continuous titration-to-effect while preserving clinician control.

command proposal module generates candidate therapeutic-device control commands based on the patient state representation and the corridor constraints. Candidate commands are proposed only when permitted by the autonomy level and are constrained to remain within corridor bounds, device capabilities, and policy-defined transition limits. In certain embodiments, the command proposal module may also generate proposals to adjust monitoring cadence as a bounded actuation used to confirm persistence, improve evidence sufficiency, or assess post-command response.

Prior to execution, candidate commands are evaluated by a constraint and safety grammar validator. The validator determines admissibility by evaluating each candidate command against a device-specific grammar profile that defines permitted command forms and prohibited parameter transitions. The grammar profile enforces semantic bounds for individual parameters and may further enforce transition constraints such as step-size limits, rate-of-change limits, or coupled constraints between multiple parameters. When a candidate command violates a constraint, the validator rejects the command and produces a reason code identifying the violated constraint. In some embodiments, the validator may optionally generate a safe-modified command or a sequence of intermediate commands that remain within admissibility constraints, subject to governance and authorization requirements.

The system further includes a risk classification and authorization module that evaluates candidate commands relative to high-risk criteria defined in the active order set or institutional policy. Based on projected outcomes, transition magnitude, or contextual factors such as signal quality, the module classifies candidate commands as low-risk or high-risk. When a command is classified as high-risk, the system prohibits dispatch unless authorization evidence is obtained from an authorized clinician. Authorization evidence may include digitally verifiable attestations, co-signatures, or other approval artifacts bound to the active order set and execution context.

validation and execution interface dispatches admissible therapeutic-device control commands to the therapeutic device only after admissibility validation and satisfaction of any required authorization evidence. The validation and execution interface associates each dispatched command with an observation window and collects post-dispatch telemetry and device acknowledgements. By enforcing secure dispatch semantics and explicit acknowledgment handling, the interface ensures that command execution is both controlled and observable.

Following dispatch, a conformance and corrective-action subsystem evaluates post-dispatch physiologic signals and therapeutic-device telemetry relative to corridor constraints. The conformance and corrective-action subsystem applies persistence gating such that transient excursions do not immediately trigger deviation conditions. A deviation condition is asserted only when nonconformance persists for a defined duration, occurs across a threshold number of consecutive samples, or satisfies a sliding-window violation criterion. This persistence logic reduces knee-jerk responses to noise or artifacts and supports safe titration in monitored environments.

When a sustained deviation condition is asserted or when runtime failures occur, a corrective-action controller selects and initiates an appropriate corrective procedure. Corrective procedures may include rollback to a prior known-safe configuration, compensatory adjustments intended to restore corridor conformance, transition of a therapeutic device to a preconfigured safe mode, or suspension of further autonomous actuation pending reauthorization. Corrective actions are subject to the same governance, admissibility validation, and authorization requirements as ordinary commands and are recorded in the audit trace.

In some embodiments, the system includes governance enforcement components that generate governance explanations and create audit records linking telemetry evidence, state estimation results, candidate commands, admissibility determinations, authorization evidence, dispatch events, acknowledgements, and post-dispatch outcomes. In exemplary implementations, execution and completion of actions may be conditioned on successful creation and append of compliant audit records to a tamper-evident store, thereby enforcing ledger-gated completion and preserving accountability.

Accordingly, the system architecture described herein provides a modular yet tightly governed framework for clinician-bounded closed-loop control of therapeutic devices. By separating observation, interpretation, proposal, validation, authorization, execution, and correction into governed components, the architecture enables safe automation while preserving clinician authority, auditability, and regulatory readiness.

### Closed-Loop Titration-to-Effect Cycle

In exemplary embodiments, the clinician-bounded autopilot assistant operates as a closed-loop titration-to-effect system in which therapeutic adjustments are performed incrementally, patient response is observed over time, and subsequent adjustments are adapted based on the observed response, all while remaining within clinician-defined corridors and governance controls. The closed-loop operation reflects established clinical titration practice and is designed to avoid abrupt or speculative actions by incorporating persistence confirmation, evidence sufficiency evaluation, and explicit escalation when uncertainty or risk is elevated.

During operation, the system continuously or periodically acquires physiologic monitoring telemetry and therapeutic-device telemetry and updates the patient state representation accordingly. The patient state representation represents both instantaneous and trend-based information and includes physiologic state variables, device status indicators, timestamps, and confidence or signal-quality indicators. When multiple telemetry sources provide overlapping measurements, the system reconciles the sources using confidence indicators, temporal alignment, and policy rules. When reconciliation fails to satisfy evidence sufficiency criteria, the system suppresses actuation and generates a governed confirmation request rather than proceeding with a potentially unsafe action.

The system compares one or more elements of the patient state representation to multi-dimensional corridor constraints obtained from the active clinician-authored order set. When a physiologic variable lies outside a corridor bound, trends toward a bound, or is projected to violate a bound based on response projection, the system evaluates whether a candidate therapeutic-device control action is permitted under the current autonomy level. Candidate actions are generated only within the permissible action classes and are constrained by device capabilities, semantic parameter bounds, and transition constraints such as step-size or rate-of-change limits.

Before asserting a deviation condition or initiating an action, the system applies evidence sufficiency and confirmation logic. The system does not treat instantaneous threshold crossings as sufficient justification for action. Instead, it evaluates whether nonconformance persists over time using persistence criteria, including defined durations, consecutive sample counts, or sliding-window violation fractions. When an excursion fails to satisfy persistence criteria, the system may increase monitoring cadence, request an additional measurement, or apply artifact rejection logic in order to improve confidence before proceeding.

In some embodiments, monitoring cadence itself is treated as a controllable actuator within the closed loop. When instability is suspected or when post-command response must be assessed with higher temporal resolution, the system may propose or dispatch a bounded cadence adjustment in accordance with the active order set and autonomy level. Such cadence changes are treated as governed actions and are subject to authorization and audit requirements analogous to therapeutic-device commands. Once stability is demonstrated for a configured duration, the system may relax monitoring cadence while recording the basis for the change and its outcomes.

When a candidate therapeutic-device control command is determined to be admissible under the device-specific safety grammar and permitted by the autonomy level, and when any required authorization evidence is satisfied, the validation and execution interface dispatches the command to the therapeutic device. The system associates the dispatch with a defined post-dispatch observation window and monitors subsequent telemetry to assess patient response. Response assessment includes evaluating whether the patient state converges toward corridor targets, overshoots bounds, oscillates, or fails to respond within an expected time-to-effect.

In certain embodiments, the system maintains a patient-specific response profile that characterizes how the patient responds to particular action classes. The response profile is updated only using governed episodes that satisfy evidence sufficiency and persistence criteria and that are traceable to executed actions and observed outcomes. By treating the patient as their own reference, the system adapts titration behavior without relying on a fixed external patient model, while preserving governance and auditability. Updates to the response profile are constrained by policy-defined limits and are recorded with order set identifiers, policy versions, and confidence indicators.

When the system detects non-convergence, repeated overshoot, oscillatory behavior, or persistent deviation despite bounded titration actions, the system escalates. Escalation may include suppressing further actuation, generating a governed tool request, requesting clinician review, requiring authorization evidence for additional actions, downgrading autonomy level, transitioning a therapeutic device to a safe mode, or suspending further actuation pending reauthorization. The escalation behavior ensures that uncertainty and risk are surfaced to the clinician rather than masked by continued automation.

The closed-loop cycle terminates when a stability criterion defined in the order set is satisfied for a configured duration, when the active order set is discontinued, when a clinician overrides or pauses autopilot operation, or when a governance policy requires suspension. Termination conditions, clinician interventions, and final patient state assessments are recorded in the governed audit trace to preserve accountability and enable post hoc review.

Accordingly, the closed-loop titration-to-effect cycle described herein provides a clinician-bounded control process that mirrors real-world clinical titration practice. The system observes, interprets, proposes, validates, executes, and adapts under explicit governance, persistence confirmation, and escalation rules, thereby enabling safe automation in regulated clinical environments without displacing clinician authority.

### Measurement Cadence Control as a Governed Actuator

In certain embodiments, the clinician-bounded autopilot assistant treats measurement cadence as an actively governed control variable rather than as a static configuration parameter. This section describes an optional embodiment of the canonical governed closed-loop operation, corresponding primarily to the Observe and Interpret stages, with cadence modification treated as a governed action within the loop, and is not required in all implementations. Monitoring cadence, including sampling frequency, cycling frequency, or polling rate of physiologic monitoring devices, is treated as a form of actuation that affects the quality, sufficiency, and timeliness of evidence used within the closed-loop control process. By governing cadence as an actuator, the system is able to dynamically increase evidence resolution during periods of instability or uncertainty and to reduce monitoring burden once stability is demonstrated, while maintaining clinician-bounded control and auditability.

The authority to modify measurement cadence is derived from the active clinician-authored order set and applicable institutional policy. In exemplary embodiments, the order set specifies whether cadence-control actions are permitted at the current autonomy level, the permissible range of cadence values, the conditions under which cadence may be increased or relaxed, and any authorization requirements associated with cadence adjustments. The system enforces these constraints at runtime such that cadence modifications outside the permitted scope are neither proposed nor executed.

During operation, the system evaluates the patient state representation, confidence indicators, projected outcome metrics, and persistence criteria to determine whether additional evidence is required to support safe titration-to-effect. When a physiologic variable trends toward or exits a corridor bound, when telemetry sources conflict, when signal quality degrades, or when post-command response must be assessed with higher temporal resolution, the system may propose or dispatch a bounded cadence adjustment. The cadence adjustment is treated as a governed action and is subjected to the same governance enforcement, authorization gating, and audit recording as therapeutic-device control commands.

In exemplary implementations, cadence-control actions are associated with a defined time window and an explicit safety purpose, such as confirming persistence of a deviation, assessing response to a therapy adjustment, or resolving uncertainty caused by conflicting or low-quality telemetry. The system records the evidence that triggered the cadence change, the corridor or policy basis for permitting the action, and the intended conditions under which cadence relaxation should occur. By associating cadence changes with explicit intent and scope, the system avoids indefinite operation at elevated monitoring intensity.

As a non-limiting illustrative example, a non-invasive blood pressure monitoring device may operate at a relatively low cycling frequency under stable conditions. When instability is suspected, such as a blood pressure value trending toward or below a lower corridor bound, the system may increase cycling frequency for a limited window in order to confirm persistence and to support safe titration. Once stability is demonstrated for a configured duration and evidence sufficiency criteria are satisfied, the system may relax the cycling frequency in one or more steps while continuing to monitor outcomes and record governed audit traces.

Cadence-control actions are subject to safety constraints designed to prevent patient discomfort, device wear, or unintended consequences. In certain embodiments, such constraints include limiting the maximum duration of high-frequency monitoring, enforcing minimum recovery intervals between high-cadence periods, reverting to default cadence upon device errors or alarms, and requiring clinician authorization evidence for extended or aggressive cadence increases. When safety constraints are violated or cannot be satisfied, the system suppresses further cadence changes and escalates to clinician review.

In exemplary embodiments, the system generates a governance explanation for each cadence-control action, identifying the triggering evidence, the applicable corridor or policy constraints, the authorization status, and the conditions for relaxation. The cadence adjustment and its outcomes are recorded in the audit trace in association with the contemporaneous patient state representation, any related therapeutic-device commands, and subsequent conformance assessments. This linkage ensures that cadence changes are transparent, explainable, and reviewable in regulated clinical environments.

Accordingly, by treating measurement cadence as a governed actuator within the closed-loop control process, the clinician-bounded autopilot assistant enhances safety and evidence quality while preserving clinician authority and accountability. Cadence-control actions become an integral part of the titration-to-effect workflow rather than an ad hoc configuration change, thereby supporting robust and auditable closed-loop operation.

### Governed Findings, Escalation, and Tool Requests

In certain embodiments, the clinician-bounded autopilot assistant generates governed findings that explicitly distinguish observed telemetry from inferred interpretations and that associate each finding with one or more confidence indicators. Governed findings serve as the primary mechanism by which the system communicates its understanding of patient and device state, determines whether bounded actuation is permitted, and decides when confirmation, escalation, or clinician intervention is required. By structuring findings under governance control, the system avoids silent assumptions and ensures that uncertainty is explicitly represented and auditable.

During operation, the system continuously evaluates incoming physiologic monitoring telemetry and therapeutic-device telemetry and transforms this data into governed findings. Each governed finding may include a description of the observed measurements, relevant trends over time, inferred state assessments, and confidence indicators reflecting signal quality, source agreement, and temporal consistency. The system explicitly separates what was directly observed from what was inferred, and identifies when an inference depends on assumptions, extrapolation, or incomplete data. Provenance metadata such as source identifiers and timestamps are retained to support traceability.

The autopilot assistant applies a closed-loop collaboration pattern in which governed findings drive the selection of a bounded next step. When evidence is sufficient and risk is low, the system may proceed with bounded actuation as permitted by the autonomy level. When evidence is insufficient, ambiguous, or conflicting, the system generates a governed tool request rather than executing an action. Tool requests are structured requests for additional evidence or intervention that must be satisfied before further autonomous actuation may occur. Such requests may involve repeating a measurement, increasing monitoring cadence for a defined window, obtaining a confirmatory laboratory or point-of-care value, verifying sensor placement or device integrity, or requesting clinician input to resolve ambiguity.

In exemplary implementations, the system generates governed queries to clinicians when it cannot maintain control within the active corridor, when response behavior fails to converge, or when risk classification indicates that additional authorization is required. These governed queries describe the current situation, summarize the supporting evidence and confidence indicators, identify any actions attempted within bounds, and specify the precise decision or confirmation required to proceed. By constraining queries to specific decision points, the system avoids vague alerts and supports efficient clinician oversight.

Escalation occurs when the system determines that continued bounded actuation is unsafe, unauthorized, or unsupported by sufficient evidence. Escalation may involve withholding execution of candidate commands, requesting explicit authorization evidence, downgrading the autonomy level, suspending further actuation, or transitioning a therapeutic device to a safe mode in accordance with policy. Escalation triggers include sustained deviation from corridor bounds despite bounded actions, detection of non-convergence or oscillatory response, conflicting telemetry that cannot be reconciled, classification of a candidate action as high-risk, failure to complete required confirmations within configured time windows, or degradation of execution or telemetry channels.

In certain embodiments, the system detects and governs omission events, which correspond to failures to perform required confirmations, reassessments, escalations, or bounded adjustments within time windows implied by the active order set or workflow policy. When an omission event is detected, the system records a governed omission finding that includes timestamps, supporting evidence, and the unmet requirement. The system may respond by downgrading autonomy, suspending actuation, generating escalation notifications, or requesting clinician review before further actions are permitted. By governing omissions as explicitly as commissions, the system addresses both unsafe actions and unsafe inaction.

The autopilot assistant supports an intern-mode communication posture in which it reports findings, uncertainty, and recommendations in a structured and explainable manner. Communications generated by the system emphasize situation awareness, supporting evidence, confidence levels, and permissible next steps within existing bounds. When multiple permissible actions exist, the system may present alternatives while identifying the relative risk or uncertainty associated with each option, leaving final selection to an authorized clinician when required.

Prior to executing any bounded action, the system generates a governance explanation that identifies the corridor constraints, policy rules, admissibility determinations, and authorization evidence that permit execution. When execution is denied or escalation is required, the governance explanation identifies the violated constraint, missing evidence, or risk criterion that prevented execution. These explanations are recorded in the audit trace and may be presented to clinicians to support transparency and accountability.

Governed findings, tool requests, escalation events, clinician responses, dispatched commands, and post-actuation outcomes are linked in a single audit trace. This linkage enables post hoc review to demonstrate that the system acted within clinician-defined corridors, surfaced uncertainty when present, escalated appropriately, and did not proceed with actuation when governance conditions were not satisfied. The audit trace thus supports both clinical accountability and regulatory compliance in environments where automated assistance interacts directly with therapeutic devices.

Accordingly, governed findings, escalation logic, and tool requests form a core safety mechanism of the clinician-bounded autopilot assistant. By explicitly representing uncertainty, enforcing confirmation before action, and structuring escalation under governance control, the system supports safe delegation of closed-loop assistance without displacing clinician judgment.

### Clinician-Bounded Autonomy Levels and Permissioning

This section describes an optional embodiment of the canonical governed closed-loop operation, corresponding primarily to the Validate/Admissibility stage, and is not required in all implementations.

In certain embodiments, the clinician-bounded autopilot assistant enforces autonomy levels that explicitly define the scope of actions the system may perform without additional clinician intervention. Autonomy levels are configured to preserve clinician authority while enabling safe delegation of bounded tasks in regulated clinical environments. Each autonomy level is associated with an active clinician-authored order set and governs whether the system may operate in a report-only posture, a recommendation posture, or a bounded actuation posture, and whether specific classes of actions require authorization evidence prior to execution.

The autonomy level functions as a policy-controlled permission boundary that constrains the system's behavior across the closed-loop cycle. The system evaluates autonomy permissions prior to proposing candidate actions, prior to dispatching commands, and prior to treating an action as completed for purposes of closed-loop progression. By enforcing autonomy at multiple stages, the system ensures that neither action proposal nor execution exceeds the authority granted by the clinician or institution.

In exemplary embodiments, autonomy levels may be configured on a per-patient, per-device, per-clinical-unit, or per-clinician-role basis, and may be updated dynamically in response to changes in patient acuity, uncertainty, or governance conditions. The system records the autonomy level in effect at the time of each proposal, execution, and escalation event, thereby preserving traceability and enabling post hoc review of whether actions were taken within the authorized scope.

Permissions within an autonomy level are represented as governed policy objects that define permitted action classes and associated scope constraints. Action classes may correspond to categories of therapeutic-device adjustments, monitoring cadence modifications, confirmation requests, escalation behaviors, or suspension actions. Scope constraints may restrict actions based on patient identity, device identity, device mode, clinician role, time windows, or evidence sufficiency criteria. The system evaluates these permissions at runtime to determine whether a candidate action is permitted, whether authorization evidence is required, or whether execution must be suppressed.

High-risk actions are treated distinctly within the autonomy framework. In exemplary embodiments, the active order set specifies one or more high-risk criteria that classify candidate actions based on factors such as transition magnitude, projected outcome metrics, or contextual risk indicators. When a candidate action is classified as high-risk, the system requires authorization evidence from an authorized clinician prior to dispatch, regardless of whether lower-risk actions are permitted at the current autonomy level. Authorization evidence is bound to the execution context and may include validity constraints such as scope limitation, expiration time, or single-use semantics.

The system supports immediate override, pause, and suspension controls that allow clinicians to halt autopilot actuation at any time. When an override or pause is initiated, the system suppresses further command dispatch and may require reauthorization or explicit clinician action to resume operation. Override and suspension events are recorded in the governed audit trace along with the basis for the action and any subsequent changes to autonomy level or order set parameters.

In certain embodiments, the system dynamically downgrades autonomy when governance dependencies fail or when evidence sufficiency criteria are not met. Such conditions may include degraded telemetry quality, conflicting measurements, ambiguous device acknowledgements, expired authorization artifacts, or unavailability of governance enforcement components. By automatically reducing autonomy under such conditions, the system prevents unsafe or unauthorized actuation and ensures that uncertainty is escalated to clinician review.

The system records autonomy configuration, permission checks, denied actions, authorization requests, and escalation events in the audit trace. Governance explanations generated by the system identify which autonomy rules and permissions controlled each decision, including why an action was permitted, denied, or gated pending authorization. This transparency supports accountability, facilitates regulatory review, and enables clinicians to understand and trust the behavior of the autopilot assistant.

Accordingly, clinician-bounded autonomy levels and permissioning provide a structured mechanism for safe delegation of closed-loop assistance. By explicitly defining what the system may do, under what conditions, and with what authorization, the system enables automation that scales clinical oversight without displacing clinician judgment or accountability.

### Patient-Specific Learning and Model Updates Under Governance

This section describes an optional embodiment of the canonical governed closed-loop operation, corresponding primarily to the Interpret and Validate stages, and is not required in all implementations.

In certain embodiments, the clinician-bounded autopilot assistant adapts to individual patient response characteristics by maintaining and updating a patient-specific response profile under explicit governance control. The response profile is not a generalized population model and does not substitute for clinician judgment. Instead, it represents a bounded characterization of how a particular patient responds to specific, clinician-permitted actions within the corridors defined by the active order set. By constraining learning to patient-specific, governed episodes, the system improves titration-to-effect behavior while preserving safety, auditability, and regulatory suitability.

The patient-specific response profile characterizes response dynamics observed during prior governed control episodes. Such dynamics may include responsiveness magnitude, time-to-effect, tendency to overshoot or oscillate, and stability margins within corridor constraints. The response profile may be indexed by action class, therapeutic-device mode, or corridor identifier so that different therapies or device configurations are characterized independently. The system treats the patient as their own reference, thereby avoiding reliance on an external or generalized patient model that could introduce uncontrolled variability.

Updates to the response profile are performed only using governed episodes that satisfy evidence sufficiency and persistence criteria. A governed episode includes telemetry evidence describing the patient state prior to action, the candidate action proposed and any admissibility determinations, authorization evidence when required, the dispatch event, post-dispatch telemetry observed over a defined window, and a conformance assessment relative to corridor constraints. By limiting updates to such traceable episodes, the system ensures that learning is grounded in high-quality, auditable evidence rather than transient artifacts or ambiguous outcomes.

Learning updates are subject to policy-defined constraints designed to prevent uncontrolled drift or unsafe adaptation. The system limits the magnitude and frequency of parameter updates, enforces minimum data requirements before applying updates, and maintains a record of prior response profile states. When subsequent outcomes indicate degraded performance, instability, or inconsistency with expected behavior, the system may revert to a previously recorded response profile state and escalate to clinician review. This rollback capability ensures that learning remains reversible and accountable.

In certain embodiments, material changes to the response profile or to derived thresholds used for risk classification require clinician review or authorization. The system may present clinicians with comparative summaries describing how updated parameters affect predicted response behavior, convergence rates, or safety margins, thereby enabling informed oversight. Higher autonomy levels that rely on updated response parameters may be enabled only after such review is completed.

Each update to the patient-specific response profile is recorded in the governed audit trace. The audit record associates the prior and updated parameter values with the governed episodes used for learning, the applicable policy identifiers, the active order set version, and associated confidence indicators. In some embodiments, audit records are cryptographically linked or otherwise protected to render post hoc modification detectable. This linkage enables later review of why a learning update occurred, what evidence supported it, and how it affected subsequent system behavior.

By maintaining patient-specific learning under governance control, the clinician-bounded autopilot assistant improves safety and effectiveness of closed-loop titration without introducing opaque or unbounded adaptation. Learning is constrained to what is clinically permissible, traceable to observed outcomes, reversible when necessary, and transparent to clinicians and regulators. Accordingly, patient-specific learning operates as a controlled enhancement to the closed-loop process rather than as an independent or unsupervised decision-making mechanism.

### Multi-modal Monitoring Inputs and Derived Hemodynamic Indices

Arterial Line Waveform Hemodynamics as a Governed Enablement Example

In certain embodiments, the clinician-bounded autopilot assistant ingests arterial pressure waveform telemetry from an invasive arterial line and computes one or more hemodynamic indices using published arterial contour or waveform analysis techniques. This section is provided as a non-limiting enablement example illustrating how waveform-derived physiologic evidence may be incorporated into the governed closed-loop framework described herein. The novelty asserted in the present disclosure does not reside in any particular arterial waveform algorithm, mathematical derivation, or physiologic model, but rather in the clinician-bounded control posture, governance enforcement, authorization gating, admissibility validation, persistence logic, and audit mechanisms that govern how such evidence is interpreted and used.

This section describes an optional embodiment of the canonical governed closed-loop operation, corresponding primarily to the Interpret stage, and is not required in all implementations.

In exemplary implementations, the sensor and telemetry interface receives a digitized arterial pressure waveform from a bedside monitor or device bridge. The waveform is treated as a time-series physiologic signal and is associated with timestamps, sampling characteristics, signal-quality indicators, and provenance metadata identifying the acquisition source and context. The system may segment the waveform into individual cardiac cycles and extract waveform features using any suitable published technique, including techniques that compute stroke volume as a function of waveform area and an impedance-like term derived from waveform morphology. Cardiac output estimates may be computed by combining stroke volume estimates with heart rate, and variability metrics may be computed over defined respiratory or temporal windows.

The system treats all waveform-derived indices as governed telemetry artifacts rather than as autonomous control signals. Each derived value is recorded with confidence indicators reflecting waveform quality, signal integrity, and the assumptions inherent in the selected computation technique. Provenance metadata identifies the specific published method or algorithmic family used to generate the index, thereby enabling transparency and post hoc review without asserting ownership or novelty over the underlying technique.

Derived hemodynamic indices are incorporated into the patient state representation only as evidence inputs and only to the extent permitted by the active clinician-authored order set and autonomy level. In exemplary embodiments, such indices are used to inform risk classification, persistence confirmation, escalation decisions, or confirmation behavior. The system does not execute therapeutic-device control actions based solely on a waveform-derived index unless such use is explicitly authorized by the order set, remains within corridor constraints, satisfies evidence sufficiency criteria, and passes admissibility and authorization gating.

When waveform-derived indices conflict with other telemetry sources or are associated with degraded signal quality, the system suppresses actuation and generates a governed finding identifying the inconsistency. In such cases, the system may request confirmation through additional measurements, increased monitoring cadence, alternative monitoring modalities, or clinician review. By explicitly governing how waveform-derived evidence is used, the system prevents over-reliance on any single analytic method and preserves clinician oversight.

In some embodiments, waveform-derived indices are used as corroborative or calibration signals rather than as primary drivers of action. For example, an estimated stroke volume or cardiac output value derived from an arterial waveform may be compared against non-invasive measurements, device telemetry, or clinician-entered values. When agreement is observed and confidence indicators are sufficient, the evidence may strengthen corridor conformance assessment. When disagreement or ambiguity persists, the system escalates rather than proceeding with actuation.

All waveform-derived indices, associated confidence indicators, and downstream uses are recorded in the governed audit trace. The audit record links the derived values to the raw telemetry window, the provenance of the computation method, the patient state representation, any resulting risk classifications or confirmation behaviors, and any clinician-facing findings or escalation events. This traceability enables later review of how waveform-derived evidence influenced system behavior without implying that the system diagnosed a condition or independently determined therapy.

Accordingly, arterial waveform analysis is presented herein as an illustrative enablement example of how complex physiologic signals may be incorporated into a clinician-bounded, governed closed-loop assistant. The system's contribution lies not in the waveform mathematics, but in the controlled, auditable, and authorization-bound manner in which such evidence is interpreted, constrained, and escalated within regulated clinical care.

### Governance Enforcement, Execution Tokens, and Ledger-Gated Completion

In certain embodiments, the clinician-bounded autopilot assistant enforces governance controls that gate both the dispatch and the completion of therapeutic-device control actions. Governance enforcement ensures that the system does not merely generate recommendations or transmit commands, but instead enforces clinician intent, institutional policy, and authorization requirements as mandatory preconditions to execution and closed-loop progression. This section describes an optional embodiment of the canonical governed closed-loop operation, corresponding primarily to the Validate/Admissibility, Authorize, and Dispatch stages, and is not required in all implementations. By binding execution to governance artifacts and auditability requirements, the system prevents unauthorized, ambiguous, or untraceable actuation in regulated clinical environments.

Prior to dispatch of any candidate therapeutic-device control command, the system evaluates whether the action is permitted by the active clinician-authored order set, the current autonomy level, and applicable governance policies. This evaluation considers corridor constraints, action-class permissions, evidence sufficiency, persistence confirmation, and high-risk classification. When the action is permitted, the system generates a governance explanation that identifies the basis for permission and enumerates any authorization evidence that is required or has been satisfied. When the action is not permitted, the governance explanation identifies the violated constraint, missing evidence, or policy condition that prevented execution, and the system suppresses dispatch.

In exemplary embodiments, the system issues an execution token that functions as a delegated and revocable authorization artifact derived from the active order set and governance policy. The execution token is bound to an execution context that may include patient identity, therapeutic-device identity, action class, corridor scope, order set version, policy identifier, permitted parameter range, and an allowable time window. The execution token limits the scope of authority for the action such that actuation is cryptographically or logically impossible without a valid token matching the execution context. Tokens may be single-use, limited-use, or time-limited, and may be invalidated upon changes to the order set, autonomy level, clinician override, or governance policy.

The validation and execution interface requires a valid execution token before dispatching any command to a therapeutic device or device bridge. If the execution token is missing, expired, scope-mismatched, or invalid, the system suppresses dispatch and records a governed failure event. By enforcing token-based dispatch, the system ensures that execution authority cannot be forged, reused outside its intended scope, or silently expanded.

In certain embodiments, the system enforces ledger-gated completion, in which a therapeutic-device control action is not treated as completed for purposes of closed-loop progression unless a compliant governed record is successfully created and appended to a tamper-evident audit store. The audit store may be implemented as an append-only, hash-linked ledger or equivalent tamper-evident structure and is not required to be distributed. The primary function of the ledger is execution control rather than data replication, such that failure to append a required record results in withholding completion acknowledgment and triggering safe behavior.

compliant governed record may associate the candidate action, the patient state representation at proposal time, the governance explanation, admissibility determinations, authorization evidence references, the issued execution token identifier, the dispatch event, device acknowledgements, and post-dispatch outcome observations. By requiring successful append of this record before completion is acknowledged, the system ensures that closed-loop progression cannot occur without durable auditability.

When ledger append fails due to unavailability, integrity check failure, or other error, the system transitions to a policy-defined safe behavior. Such behavior may include suspending further autonomous actuation, downgrading autonomy to report-only mode, escalating to clinician review, or maintaining current device settings until governance dependencies are restored. The system records the failure and the resulting safe behavior in a governed failure record to preserve traceability.

In exemplary embodiments, governance enforcement also applies to runtime failure modes unrelated to ledger availability. Such failure modes may include ambiguous device acknowledgements, negative acknowledgements, command timeouts, device bridge unavailability, or conflicting telemetry that prevents outcome assessment. When such conditions occur, the system suppresses further actuation, records a governed failure event, and escalates to clinician review rather than attempting speculative recovery.

Governance enforcement links the full execution path into a single trace that spans observation, interpretation, proposal, validation, authorization, dispatch, acknowledgement, outcome assessment, and any learning updates applied to patient-specific response profiles. This trace linkage enables post hoc review demonstrating that actions were taken only within clinician-defined corridors, that high-risk actions were properly authorized, and that uncertainty or failures resulted in escalation rather than silent continuation.

Accordingly, execution tokens and ledger-gated completion transform governance from a passive documentation mechanism into an active execution control layer. By making authorization and auditability mandatory preconditions to both dispatch and completion, the clinician-bounded autopilot assistant enforces accountability, prevents unsafe automation, and supports deployment in regulated clinical environments where traceability and control are paramount.

### Closed-Loop Corrective Actions: Rollback, Compensate, Safe-Mode, Suspend

In certain embodiments, the clinician-bounded autopilot assistant includes a closed-loop corrective-action capability that responds to sustained nonconformance, execution failures, or unsafe operating conditions by selecting and initiating preconfigured corrective procedures under governance control. Corrective actions provide a safety backstop for bounded actuation by enabling the system to recover from undesirable outcomes without resorting to uncontrolled or speculative behavior, while preserving clinician authority and auditability.

The system asserts a deviation condition only after applying persistence and confirmation criteria that distinguish transient excursions from sustained nonconformance. When a deviation condition is asserted, the corrective-action controller evaluates the current patient state, recent command history, device status, and applicable policy constraints to determine whether a corrective procedure is warranted. Corrective procedures are not initiated in response to instantaneous threshold crossings or isolated telemetry artifacts, thereby reducing the risk of knee-jerk reactions in safety-critical environments.

Corrective procedures are preconfigured and stored under governance control and are selected based on applicability conditions that account for therapeutic-device type, device mode, command class, deviation class, and autonomy level. In exemplary embodiments, a rollback procedure restores one or more device parameters to a prior configuration that was previously recorded as known-safe under the active order set. A compensatory procedure issues an offsetting or stabilizing adjustment intended to reduce overshoot, damp oscillation, or return the patient toward corridor conformance without undoing the full prior action. A safe-mode procedure transitions a therapeutic device to a preconfigured safe configuration defined by device policy or institutional guidelines. A suspension procedure prevents further autonomous actuation while allowing continued monitoring, governed reporting, and clinician intervention.

In some embodiments, the system associates one or more corrective procedures with a candidate therapeutic-device control command prior to dispatch based on a projected outcome metric or expected response. By precomputing contingency associations, the system is able to initiate an appropriate corrective procedure promptly when a sustained deviation condition is detected, while still applying governance checks and admissibility validation. This anticipatory association improves response time without bypassing authorization or safety controls.

Corrective actions are executed through the same validation and execution interface used for ordinary therapeutic-device control commands and are subject to the same governance enforcement, admissibility validation, and authorization requirements. When a corrective procedure involves a high-risk transition, requires departure from standard corridors, or affects multiple device parameters, the system may require authorization evidence from an authorized clinician prior to execution. When authorization cannot be obtained or governance dependencies are unavailable, the system defaults to a policy-defined safe behavior that suppresses further actuation and escalates to clinician review.

The system records each corrective action in the governed audit trace, linking the action to the deviation condition that triggered it, the persistence criteria satisfied, the patient state representation at the time of selection, the applicable policy identifiers, and any authorization evidence obtained. Post-execution telemetry is observed to assess whether the corrective action restored corridor conformance, stabilized the patient state, or necessitated further escalation.

In certain embodiments, corrective actions may also be triggered by runtime failures unrelated to physiologic nonconformance. Such failures may include device communication errors, ambiguous acknowledgements, execution token invalidation, or ledger-gated completion failures. In these cases, the system applies corrective behavior consistent with policy, which may include reverting to a prior configuration, transitioning to safe mode, or suspending autonomous actuation until governance dependencies are restored.

By integrating corrective actions into the closed-loop control architecture, the clinician-bounded autopilot assistant ensures that bounded actuation remains reversible, explainable, and accountable. Corrective actions are not treated as exceptions or overrides, but as governed responses that preserve patient safety and clinician oversight when deviations or failures occur.

### Example Clinical Scenarios (Non-Limiting)

In certain embodiments, the clinician-bounded autopilot assistant is applied to common clinical scenarios to illustrate how the governed closed-loop control framework operates in practice. The scenarios described herein are provided as non-limiting examples intended to demonstrate operability, safety, and governance behavior of the system across different therapeutic contexts. These examples do not define or restrict the scope of the claims and are not intended to imply that any particular therapy, device, or clinical setting is required.

In an exemplary implementation involving vasopressor titration to maintain a target mean arterial pressure corridor, a clinician activates an order set that defines a desired physiologic target, permissible corridor bounds, allowable infusion parameters, and step-size and rate-of-change limits. The system observes physiologic telemetry including blood pressure and heart rate and updates the patient state representation with associated confidence indicators. When the mean arterial pressure trends toward or below a lower corridor bound, the system evaluates persistence criteria to distinguish transient artifacts from sustained nonconformance. When persistence criteria are satisfied and evidence sufficiency is met, the system proposes a bounded infusion adjustment. If the proposed adjustment is classified as low-risk under the active order set and permitted by the autonomy level, the system may dispatch the adjustment under governance control. When classified as high-risk, the system suppresses dispatch and requests authorization evidence prior to execution. During instability, the system may increase blood pressure measurement cadence as a governed actuator to confirm persistence and assess response, and later relax cadence as stability is demonstrated. All findings, proposals, authorizations, dispatch events, acknowledgements, and outcomes are recorded in a tamper-evident audit trace.

In another exemplary implementation involving ventilator parameter adjustment within bounded settings, a clinician defines corridor constraints for parameters such as tidal volume, inspiratory pressure, respiratory rate, or fraction of inspired oxygen. The system observes ventilator telemetry and patient physiologic response and generates governed findings describing conformance and trends. Candidate parameter adjustments are proposed only within permitted bounds and are validated using a device-specific safety grammar that enforces semantic limits and transition constraints. When a proposed adjustment is admissible and permitted, the system may dispatch the command through a validation and execution interface. When non-convergence, overshoot, or sustained deviation is detected, the system escalates to clinician review and may initiate a corrective action such as rollback to a prior configuration, transition to a safe mode, or suspension of further autonomous actuation pending reauthorization.

In an exemplary implementation involving insulin infusion management with continuous glucose monitoring, the system receives interstitial glucose measurements and associated confidence indicators from a transcutaneous sensor. The system evaluates glucose trends over time and incorporates the measurements into the patient state representation. When a glucose value trends outside a clinician-defined corridor, the system evaluates persistence and evidence sufficiency prior to proposing a bounded insulin adjustment. When confidence is degraded or trends are discordant, the system suppresses actuation and requests a confirmatory point-of-care measurement. When a proposed adjustment is classified as high-risk, the system requires clinician authorization evidence prior to dispatch. Glucose measurements, proposals, authorization events, and post-adjustment outcomes are recorded as governed artifacts.

In another exemplary implementation involving sedation or analgesia management, the system applies corridor constraints to physiologic proxies such as respiratory rate, end-tidal carbon dioxide, or other monitored indicators of sedation depth. When telemetry suggests a deviation from the desired corridor, the system generates governed findings and evaluates whether bounded titration is permitted. When waveform morphology or signal quality suggests obstruction, rebreathing, or device-related artifacts, the system suppresses actuation and generates a governed tool request for confirmation or troubleshooting rather than proceeding with escalation. This report-first posture ensures that automation does not amplify risk when evidence is ambiguous.

In certain embodiments, the system coordinates bounded adjustments across multiple therapeutic devices under coupled constraints. For example, adjustments to ventilator settings may be gated when hemodynamic indices indicate preload sensitivity, or vasopressor titration may be constrained when fluid balance data suggests hypovolemia. In such scenarios, the system enforces coupled constraints through safety grammar validation and risk classification, and escalates to clinician review when competing objectives cannot be reconciled within permitted bounds.

These scenarios illustrate how the clinician-bounded autopilot assistant observes physiologic and device telemetry, interprets evidence under governance control, proposes or executes only those actions that are explicitly authorized, escalates when uncertainty or risk is elevated, and maintains a comprehensive audit trail. The scenarios are provided to demonstrate practical operation and do not limit the invention to any specific clinical use case or device configuration.

### Interoperability, Device Bridges, and Clinical System Integration

In certain embodiments, the clinician-bounded autopilot assistant interoperates with heterogeneous therapeutic devices, monitoring systems, and clinical information systems through one or more governed integration layers. Interoperability is treated as a governance boundary rather than as a transparent data conduit, such that policy enforcement, admissibility validation, authorization gating, and safe failure behaviors are applied whenever telemetry or commands cross system boundaries. This design prevents silent degradation, uncontrolled actuation, or loss of accountability when external systems behave unexpectedly, provide inconsistent data, or become unavailable.

In exemplary implementations, the system includes a device bridge or adapter layer configured to communicate with therapeutic devices and monitoring devices using vendor-specific protocols, middleware interfaces, or standardized clinical communication formats. The device bridge converts inbound telemetry, alarms, and status indicators into a governed internal representation that includes timestamps, provenance metadata, and signal-quality indicators. Similarly, the device bridge translates governed command intents generated by the autopilot control engine into device-specific command messages, while preserving parameter bounds, transition constraints, and execution context enforced by the governance layer.

The device bridge preserves acknowledgment and completion semantics for therapeutic-device control commands. The system receives explicit device acknowledgements indicating acceptance, rejection, execution, timeout, or ambiguous completion states and treats such acknowledgements as governed telemetry. When acknowledgements are negative, delayed, ambiguous, or inconsistent with expected behavior, the system records a governed failure event, withholds completion acknowledgment, and transitions to a policy-defined safe behavior, such as suspending further actuation or escalating to clinician review. This behavior prevents silent command failure and preserves closed-loop safety in the presence of unreliable or degraded device interfaces.

Prior to transmitting any command across an integration boundary, the system enforces governance requirements including validation of execution authorization artifacts, scope and parameter constraints, admissibility under device-specific safety grammar profiles, and satisfaction of any required authorization evidence. In some embodiments, the device bridge performs an additional validation step to ensure that translated device-specific commands remain within permitted bounds. When translation fails, validation does not pass, or communication channels are degraded, the system suppresses dispatch and generates a governed explanation describing the failure rather than attempting speculative recovery.

In exemplary embodiments, an execution authorization artifact encodes a bounded execution scope including at least a patient identifier, a therapeutic-device identifier, an authorized action class, an applicable corridor identifier, one or more permitted parameter ranges or transition constraints, and an allowable time window during which the action may be dispatched.

In certain embodiments, the clinician-bounded autopilot assistant integrates with electronic medical record systems to ingest clinician-authored orders, order sets, corridor definitions, autonomy configurations, and policy identifiers. The system may also ingest structured clinical observations, laboratory results, and clinician-entered measurements from the medical record to enrich the patient state representation and support evidence sufficiency determination. Integration with the medical record does not bypass governance controls; rather, imported data is treated as telemetry with provenance and confidence indicators and is subject to the same confirmation, validation, and escalation logic as device-generated data.

In exemplary implementations, the system may write back governed artifacts to the electronic medical record or associated clinical systems. Such artifacts may include governed findings, recommendations, audit summaries, or references to executed actions and may be associated with encounter identifiers, clinician identifiers, order set versions, and policy identifiers to preserve traceability. Write-back operations are governed and, in some embodiments, are limited to report-first outputs rather than executable orders, thereby preserving clinician oversight and institutional workflows.

In some embodiments, the system receives derived metrics computed by third-party or closed systems, such as hemodynamic indices, waveform-derived measures, or decision-support outputs. Such imported metrics are treated as governed telemetry artifacts and are recorded with provenance metadata identifying the source system, computational method, and timestamp. The system may use imported metrics as corroborative evidence or confirmation inputs for governed findings, risk classification, or escalation behavior, while maintaining a report-first posture unless the active clinician-authored order set expressly permits bounded actions based on such metrics.

Integration with external systems is performed under explicit safe failure semantics. When telemetry feeds, device bridges, network connections, or electronic medical record interfaces are unavailable, degraded, or inconsistent, the system transitions to policy-defined safe behaviors. Such behaviors may include downgrading autonomy to a report-only posture, suspending command dispatch, requesting confirmatory measurements from alternative sources, escalating to clinician review, or recording a governed integration failure event. These behaviors ensure that loss of connectivity or data integrity does not result in unsafe actuation or silent degradation of control.

In exemplary implementations, the system supports offline buffering and later reconciliation of telemetry and governed records when connectivity disruptions occur. Telemetry and audit records may be buffered locally and later reconciled with a central audit store when connectivity is restored, while actuation authority is restricted during the disruption in accordance with policy. This approach preserves audit integrity and traceability without sacrificing patient safety.

Interoperability is designed to accommodate variability in device capabilities, communication protocols, data schemas, and institutional workflows. The system supports adapter-based integration and schema mapping to normalize heterogeneous inputs and outputs while preserving execution-path enforcement, authorization gating, and ledger-gated completion. Accordingly, the interoperability mechanisms described herein allow the clinician-bounded autopilot assistant to operate across heterogeneous devices and clinical systems while maintaining governance, safety, and accountability in complex, multi-vendor clinical environments.

### Clinical Assistant Workflow Orchestration Under Governance Control

In certain embodiments, the clinician-bounded autopilot assistant further comprises a workflow orchestration component configured to coordinate human-in-the-loop tasks, documentation assistance, and notifications as part of the governed closed-loop operation. The workflow orchestrator operates as an extension of the governed control system rather than as a separate clinical workflow tool, ensuring that human interactions are treated as governed events that affect the system's permitted action space and closed-loop progression.

The workflow orchestrator receives governed events generated by the autopilot control engine, including interpreted findings, corridor nonconformance determinations, candidate action proposals, admissibility determinations, risk classifications, authorization requests, execution acknowledgements, deviation conditions, corrective actions, and omission detections. Based on these events, the workflow orchestrator generates corresponding workflow actions—such as tasks, prompts, or notifications—that are explicitly tied to governance requirements and policy conditions rather than to generic task scheduling.

In exemplary implementations, prior to execution of a bounded action, the workflow orchestrator presents a governance explanation that summarizes the applicable order set identifier, corridor constraints, triggering evidence, proposed action class, and any authorization evidence required. This presentation enables clinicians to understand why an action is being proposed, gated, or denied and to provide informed authorization, confirmation, or modification of the active order set when appropriate. When an action is not permitted, the workflow orchestrator may present an explanation identifying the violated constraint, missing evidence, or unmet authorization requirement.

When evidence sufficiency is not satisfied or when telemetry sources conflict, the workflow orchestrator generates governed task requests for confirmation rather than permitting actuation. Such tasks may include requests to repeat a measurement, increase monitoring cadence for a defined window, verify sensor placement or device integrity, obtain confirmatory laboratory values, or request clinician input to resolve ambiguity. The workflow orchestrator tracks task creation, deadlines, and completion evidence and associates task resolution with subsequent closed-loop decisions.

The workflow orchestrator maintains a governed encounter task and event state that tracks required confirmations, reassessments, escalation steps, authorization requests, and documentation obligations associated with the active order set and autonomy level. Task and event status is updated based on clinician acknowledgements, receipt of confirmatory measurements, device acknowledgements, and successful append of governed audit records. When required tasks or authorizations are not completed within policy-defined time windows, the workflow orchestrator causes the system to downgrade autonomy, suspend actuation, or escalate to clinician review, while generating a governance explanation identifying the unmet condition.

In certain embodiments, the workflow orchestrator detects omission events corresponding to failures to perform required confirmations, reassessments, escalations, or documentation actions within configured time windows implied by the active order set, corridor policy, or workflow plan. Upon detecting an omission, the system records a governed omission finding, applies policy-defined safe behavior—such as downgrading autonomy or suspending further actuation—and escalates to clinician review. This behavior ensures that unsafe inaction is governed and auditable rather than silently ignored.

In exemplary implementations, the workflow orchestrator supports documentation assistance under governance control. The system may generate structured summaries or draft clinical notes that associate interpreted observations, decision rationale, authorization evidence, and outcomes with order set versions, policy identifiers, and timestamps. Such documentation artifacts are explicitly identified as drafts or suggestions and require clinician review prior to finalization, thereby preserving clinician authorship, judgment, and accountability.

Notifications generated by the workflow orchestrator are context-sensitive and governance-aware. Notifications may be directed to authorized clinician interfaces and may include escalation alerts, authorization requests, omission notifications, corrective-action notices, or reminders to restore temporary configuration changes such as monitoring cadence adjustments. Each notification is associated with a governed explanation and is recorded in the audit trace to preserve accountability and to support later review of clinician-system interaction. Notification delivery may incorporate escalation pathways and timing controls to reduce alert fatigue.

In some embodiments, the workflow orchestrator supports governed write-back of summaries or structured data to clinical systems, including electronic medical records. Such write-back operations may include references to governed findings, action outcomes, authorization evidence, or audit summaries and may be restricted by role-based access controls and institutional policy. Write-back is performed in a manner that preserves separation between governed assistance and final clinical decision-making and does not bypass clinician review.

Closed-loop progression—including issuance of subsequent execution authorization artifacts or dispatch of additional actions—may be conditioned on resolution of relevant workflow tasks, confirmations, or authorizations. By integrating human responses as explicit control inputs, the workflow orchestrator ensures that clinician interactions are incorporated into the governed closed-loop process rather than treated as external interruptions or informal side channels.

Accordingly, the clinical assistant workflow orchestrator enables structured collaboration between the clinician-bounded autopilot assistant and human clinicians. By coordinating tasks, confirmations, documentation, and notifications under governance control, the system preserves clinician authority while enabling safe, auditable delegation of monitoring, titration, escalation, and documentation activities in regulated clinical environments.

### Implementation Variations and Non-Limiting Embodiments

In certain embodiments, the clinician-bounded autopilot assistant may be implemented using a variety of computing architectures, deployment models, policy representations, and analytic techniques without departing from the scope of the claimed invention. The embodiments described herein are intended to illustrate operability and enablement rather than to limit the invention to any particular hardware arrangement, software stack, clinical guideline, or institutional workflow.

The system may be deployed in different physical and logical configurations depending on clinical setting and operational requirements. In some embodiments, components of the autopilot assistant are deployed at the bedside as a local appliance or embedded system, while other components operate on an on-premises server, a cloud-based platform, or a hybrid combination thereof. Telemetry ingestion, governance enforcement, secure execution, and audit retention may be partitioned across edge and centralized components, provided that governance controls, authorization gating, and safe failure behaviors are preserved.

The internal logic used to interpret telemetry and propose candidate actions may vary across embodiments. In some implementations, interpretation relies on deterministic rules or protocol-based logic derived from clinical guidelines. In other implementations, statistical models, adaptive estimators, or machine-learning components are used to generate governed findings or candidate actions. Regardless of the internal analytic technique, all outputs remain subject to corridor constraints, admissibility validation, authorization gating, and governance enforcement prior to any actuation. The invention does not depend on any particular analytic method, and no claim is limited to the use of a specific model or algorithm.

Policy representations may also vary across embodiments. Clinician-authored order sets, corridor constraints, authorization rules, and device safety grammars may be represented as structured templates, rulesets, graphs, or other policy artifacts. In some embodiments, policies are represented as a policy graph that encodes permissions, dependencies, and escalation pathways, and the governance layer evaluates applicability by traversing the policy graph based on patient context, device identity, clinician role, and current autonomy level. Variations in policy representation do not alter the requirement that actions remain clinician-bounded and auditable.

The system may support multiple data schemas and adapter interfaces to accommodate heterogeneity among medical devices, monitoring systems, and electronic medical record platforms. Inbound data may be normalized into a governed internal schema, and outbound commands may be translated into device-specific formats, while preserving execution-path enforcement and ledger-gated completion. Differences in vendor protocols or data formats do not affect the core operation of the invention so long as governance, authorization, and admissibility controls are maintained.

Safety validation techniques may also vary across embodiments. Admissibility validation may be implemented using device-specific safety grammar profiles, semantic bounds, transition constraints, or combinations thereof. Outcome evaluation may rely on bounded response estimation, patient-specific response profiles, or conservative rule-based checks rather than on external population models. In all cases, outcome evaluation is used to support governance, risk classification, and escalation rather than to independently determine therapy outside clinician-defined corridors.

The embodiments described herein may be combined, modified, or selectively implemented without departing from the scope of the claims. Features described in one embodiment may be used in conjunction with features described in another embodiment, and no single embodiment is required to practice the invention. The invention is defined by the claims and their equivalents, and the foregoing description is intended to provide enabling disclosure and context without imposing unnecessary limitations.

### Conceptual Framework and Interpretive Context (Non-Limiting)

This subsection provides non-limiting conceptual context for understanding how the clinician-bounded closed-loop assistant operates as described in this specification. The descriptions herein are intended to aid comprehension of the system's governance posture and operational philosophy and are not intended to serve as formal definitions for claim construction, which are provided elsewhere in this disclosure.

As described throughout this specification, the clinician-bounded assistant operates under a governance model in which interpretation, recommendation, and actuation are explicitly constrained by clinician-authored intent. The assistant is designed to operate within boundaries defined by active order sets, autonomy configurations, and corridor policies, and does not function as an independent clinical decision maker. When uncertainty, risk, or insufficient evidence is detected, the system favors escalation, confirmation, or report-only behavior rather than unbounded action.

In this context, corridor-based control expresses the concept that acceptable physiologic states and device behaviors are bounded by clinician-defined limits rather than by fixed targets or algorithmic optimization. These bounds may be multi-dimensional, temporally conditioned, or dynamically revised by the clinician, and they serve to constrain what the system may propose or execute at any given time.

Similarly, autonomy within the system is conceptualized as a permission state rather than a level of intelligence or independence. Autonomy governs which classes of actions may proceed without additional clinician involvement and may be raised, lowered, or suspended in response to patient state, evidence quality, institutional policy, or clinician input. Autonomy therefore reflects governance posture rather than clinical authority.

The system's use of evidence, confidence indicators, and persistence criteria reflects an intent to distinguish reliable signals from transient artifacts and to avoid premature actuation. Rather than assuming correctness of any single measurement or model output, the system evaluates sufficiency and consistency of available evidence before progressing along the closed-loop execution path.

Governance explanations and audit mechanisms are employed to ensure that actions taken or withheld by the system can be understood, reviewed, and traced to the applicable policies and clinical intent. These mechanisms support transparency and accountability without requiring disclosure of internal policy logic or proprietary implementation details.

Overall, this conceptual framework is intended to clarify how the disclosed embodiments preserve clinician authority, bounded automation, and regulatory accountability while enabling safe closed-loop assistance. The concepts described in this subsection are illustrative and contextual, and no requirement that any embodiment include all described features is implied unless expressly recited in the claims.

### Additional Embodiments, Variations, and Extensions

The clinician-bounded autopilot assistant described herein may be implemented in a wide variety of embodiments, deployment forms, and technical configurations without departing from the scope of the claimed invention. The embodiments described throughout this specification are illustrative rather than exhaustive, and features described in one embodiment may be combined with features described in other embodiments unless expressly stated otherwise. The invention is not limited to any particular hardware architecture, software stack, policy representation, or institutional workflow.

In certain embodiments, the system is implemented using different data schemas, policy formats, cryptographic mechanisms, adapter interfaces, or device command grammars. These implementation choices may vary across institutions, device vendors, or regulatory jurisdictions, while preserving the core operational posture of clinician-bounded assistance with governance enforcement, admissibility validation, authorization gating, and auditability. The ability to substitute or adapt these components ensures interoperability and deployability across heterogeneous clinical environments.

In some embodiments, the autopilot assistant supports multiple operational modes, including advisory-only operation, supervised closed-loop assistance, and bounded autonomous operation subject to policy and authorization rules. The selection among these modes may depend on patient acuity, clinical setting, device class, or clinician preference, and may change dynamically during an encounter. Regardless of mode, the system maintains a report-first posture when uncertainty is elevated or evidence sufficiency is not satisfied, and refrains from actuation unless explicitly permitted.

In certain embodiments, the system coordinates actions across multiple therapeutic devices associated with a single patient. In such configurations, corridor constraints, safety grammar profiles, and escalation rules may be applied jointly across devices to enforce coupled safety conditions. For example, permissible adjustments to one device parameter may depend on the current or projected state of another device or physiologic variable. These coupled constraints are enforced under governance control to prevent unsafe interactions while enabling coordinated titration-to-effect.

In some embodiments, updates derived from stored artifacts, including patient-specific response profiles, policy thresholds, or corrective procedure applicability conditions, are versioned and auditable. Such updates may be associated with policy identifiers, corridor identifiers, order set identifiers, device configuration versions, and timestamps. The system may restrict the ability to apply updates to authorized roles and may require additional approvals for updates affecting high-risk actions, while preserving the ability to revert to prior versions if adverse effects are detected.

In certain embodiments, the system generates feedback artifacts that capture the relationship between observed patient state, proposed or executed actions, authorization evidence, and post-dispatch outcomes. These artifacts may be stored under governance control and used to support supervised learning, evaluation, or refinement of bounded response parameters. Importantly, such updates do not create uncontrolled autonomy; rather, they remain constrained by clinician-defined corridors, autonomy levels, and policy rules, with all changes recorded in an auditable manner.

In some embodiments, the assistant incorporates additional signal interpretation capabilities, including waveform analysis, trend detection, or pattern recognition, to enrich governed findings and escalation logic. These capabilities may be implemented using rule-based logic, statistical methods, machine learning models, or combinations thereof. Regardless of the internal method used, outputs are treated as evidence with associated confidence indicators and provenance, and are subject to the same governance and report-first constraints described elsewhere in this specification.

In certain embodiments, the system supports deployment-level governance and application surveillance. Software components may be versioned, registered, and associated with policy identifiers, and execution may be conditioned on an allowed application-version list and device-profile compatibility. The system may monitor runtime conformance metrics, such as acknowledgment failures, deviation rates, policy denials, or rollback frequency, and may automatically downgrade autonomy, increase confirmation behavior, or suspend actuation when abnormal patterns are detected, while preserving auditability by associating actions with version identifiers and policy versions.

These variations and extensions do not alter the fundamental posture of the invention. Across all embodiments, the clinician-bounded autopilot assistant listens to monitoring and device telemetry, interprets data under governance control, proposes or executes only those actions that are authorized within clinician-defined corridors, escalates when uncertain or high-risk, and maintains auditability suitable for regulated clinical environments. The described embodiments are therefore intended to illustrate breadth and flexibility of implementation rather than to limit the scope of the claims.

### Capnography Waveform Interpretation and Pattern Recognition for Governed Closed-Loop Safety

In certain embodiments, the clinician-bounded autopilot assistant incorporates capnography waveform interpretation as a governed safety and context signal within the closed-loop control framework. Capnography data is treated as a feature-rich physiologic signal rather than merely a numeric end-tidal carbon dioxide value, and is used to inform confidence assessment, risk classification, confirmation behavior, and escalation logic under governance control. Unless expressly authorized by an active clinician-authored order set and autonomy level, capnography-derived information is used in a report-first posture to gate or suppress actuation rather than to independently drive therapeutic-device control.

In exemplary implementations, the sensor and telemetry interface receives capnography data comprising a time-series capnogram waveform, an end-tidal carbon dioxide value, an inspired carbon dioxide value, and associated metadata including timestamps, sampling characteristics, and signal-quality indicators. The state estimation module processes the capnogram waveform to extract waveform-shape features that characterize the phases of the respiratory cycle, including baseline behavior, expiratory upstroke morphology, alveolar plateau characteristics, and inspiratory downstroke behavior. These features may include, without limitation, slope, duration, curvature, angle relationships between phases, breath-to-breath variability, and artifact indicators reflecting sampling line integrity or signal distortion.

In certain embodiments, the system computes a capnography feature vector from the waveform and applies a classification process to identify one or more waveform pattern classes that are relevant to closed-loop safety and governance. Such pattern classes are not required to constitute a clinical diagnosis. Rather, they serve as signal-quality, safety, and control-context indicators used to determine whether autonomous actuation is permitted, whether confirmation behavior is required, or whether escalation to clinician review is necessary. By explicitly separating waveform interpretation from diagnosis, the system preserves a clinician-bounded posture and avoids ungoverned clinical inference.

In exemplary implementations, the system may identify waveform patterns consistent with absent or near-zero exhaled carbon dioxide, abrupt loss of waveform amplitude, elevated inspiratory baseline suggestive of rebreathing or apparatus mixing, obstructive expiratory morphology indicative of airflow limitation, plateau cleft patterns suggestive of spontaneous respiratory effort or circuit integrity issues, and oscillatory patterns associated with cardiogenic oscillations or waveform perturbations. Each identified pattern is recorded as a governed finding with associated confidence indicators, provenance metadata, and references to the applicable order set and policy identifiers.

When a waveform pattern indicates elevated risk or degraded reliability of capnography measurements, the risk classification and authorization module may classify the current state as high-risk for purposes of closed-loop actuation. In such cases, the system suppresses autonomous therapeutic-device control actions, increases monitoring cadence if permitted, and initiates confirmation behavior or escalation. Confirmation behavior may include requesting verification of airway placement, checking circuit integrity, obtaining corroborating physiologic measurements, or requesting clinician confirmation before further actuation is considered. These behaviors are governed actions that are recorded and linked to the triggering evidence.

In certain embodiments, the capnography feature vector is incorporated into the patient state representation and influences evidence sufficiency determination. When waveform quality is degraded, when features are inconsistent with other telemetry, or when classification confidence is low, the system treats evidence as insufficient and refrains from actuation. In exemplary implementations, the system explicitly reports the uncertainty and the basis for suppression or escalation to an authorized clinician interface, rather than attempting to compensate silently or extrapolate beyond the available evidence.

In some embodiments, capnography waveform interpretation is used to dynamically adjust closed-loop safety constraints. For example, when obstructive morphology is detected, the system may restrict or prohibit certain command classes that would increase risk in the presence of airway obstruction, require additional authorization evidence, or mandate confirmation measurements prior to further titration. When spontaneous respiratory effort is inferred, the system may downgrade autonomy level or request clinician review before executing ventilator adjustments that could interfere with patient-initiated breathing.

In exemplary implementations, capnography-derived findings are integrated with other physiologic signals, including airway pressure, tidal volume, respiratory rate, oxygen saturation, and device telemetry, to support cross-validation and conflict detection. When capnography findings conflict with other telemetry sources, the system generates a governed finding describing the inconsistency and initiates confirmation or escalation behavior rather than proceeding with actuation. This multi-modal reconciliation supports safe operation in environments where individual sensors may be unreliable or intermittently degraded.

In certain embodiments, the system applies capnography-based gating as part of closed-loop safety rather than closed-loop control. That is, capnography interpretation is used primarily to determine whether it is safe to proceed with otherwise permissible actions, to increase monitoring fidelity, or to suspend actuation, rather than to directly compute therapeutic-device parameter changes. This design choice preserves clinician authority and aligns capnography use with its role as a safety and monitoring modality in regulated care.

In exemplary implementations, all capnography-derived features, pattern classifications, confidence indicators, and resulting gating or escalation decisions are recorded as governed artifacts in the audit trail. These records may include references to waveform segments, extracted feature values, classification confidence, applied policy identifiers, and subsequent actions taken or withheld. The audit trail enables post hoc review of how capnography evidence influenced system behavior and supports accountability in regulated environments.

Accordingly, the incorporation of capnography waveform interpretation within the clinician-bounded autopilot assistant enhances closed-loop safety by providing rich, governed evidence about ventilation, airway integrity, and measurement reliability, while preserving a report-first posture and ensuring that therapeutic-device actuation occurs only when authorized, supported by sufficient evidence, and consistent with clinician-defined corridors and governance controls.

### Operational Advantages and Clarifying Context for Regulated Clinical Deployment (Non-Limiting)

The systems and methods described herein provide a clinician-bounded closed-loop assistant that is specifically structured for operation in regulated clinical environments. The assistant is designed to listen to physiologic monitoring data and therapeutic-device telemetry, interpret such data under governance control, and propose or execute actions only within clinician-defined corridors, autonomy levels, and authorization requirements. This posture distinguishes the disclosed embodiments from generic automation or advisory systems and reflects the practical realities of regulated care delivery.

In exemplary implementations, the assistant does not replace clinician judgment and does not operate as an unsupervised autonomous clinician. Instead, it functions as a governed bedside execution assistant that performs delegated tasks analogous to those performed by trained clinical staff operating under physician orders. When uncertainty is elevated, evidence is insufficient, or a candidate action is classified as high-risk, the system refrains from actuation and escalates to clinician review. This design preserves clinician authority while enabling safe delegation of bounded, repetitive, or time-sensitive tasks.

The disclosed embodiments combine multiple safeguards that collectively enable closed-loop assistance without unbounded autonomy. These safeguards include clinician-authored corridor constraints that define permissible physiologic states and device parameters, autonomy levels that restrict which action classes may be executed, persistence gating to prevent knee-jerk responses to transient artifacts, and device-specific safety grammar validation that enforces semantic and transition constraints on commands. Corrective procedures such as rollback, compensatory action, safe-mode transitions, and suspension further provide safety backstops when sustained deviation or runtime failures occur.

Governance enforcement is integrated directly into the execution path rather than treated as an external audit function. In exemplary implementations, proposed actions are evaluated against policy and order set constraints prior to dispatch, authorization evidence is required for high-risk actions, and execution authority is bound to a specific clinical context through execution tokens. Completion of actions may be conditioned on successful creation of tamper-evident audit records, ensuring that actions are not silently completed without accountability. These mechanisms support traceability and post hoc review, which are critical in regulated environments.

The assistant further supports confirmation behavior and evidence sufficiency checks to reduce false alarms and inappropriate actuation. Rather than responding to instantaneous threshold crossings, the system applies persistence criteria and may adjust monitoring cadence to confirm whether a deviation is sustained. When telemetry sources conflict or signal quality is degraded, the system surfaces uncertainty to clinicians and requests additional evidence rather than extrapolating beyond available data. This behavior aligns with established clinical practice and reduces alarm fatigue and automation bias.

Patient-specific response learning is incorporated in a bounded and auditable manner. In exemplary implementations, the system learns how an individual patient responds to bounded actions over time and uses this information to refine low-risk titration steps and response projections. Such learning is constrained by policy, versioned, and linked to supporting evidence, and does not introduce uncontrolled model drift or autonomous goal formation. The patient remains their own reference, and learning occurs within clinician-defined corridors.

The described embodiments also define explicit safe behaviors for runtime failures, including degraded device connectivity, ambiguous acknowledgements, expired authorization artifacts, or governance dependency failures. In such cases, the system defaults to a deny-by-default posture for actuation, downgrades autonomy as appropriate, and escalates to clinician review. This approach reduces the risk of silent failure and aligns with safety expectations in clinical environments where infrastructure dependencies may be imperfect.

Taken together, these characteristics establish a clinician-bounded assistant that enables closed-loop titration-to-effect under governance control, rather than an unregulated autonomous system. The assistant listens, interprets, proposes, and, when permitted, acts within defined corridors, while maintaining explainability, authorization, and auditability. The disclosed systems and methods therefore provide a practical and deployable framework for enhancing clinical efficiency and safety without displacing clinician responsibility.

The foregoing discussion is intended to provide clarifying context for understanding the disclosed embodiments and their operation in regulated care settings. It is not intended to limit the scope of the claims or to characterize any claim element as required unless expressly recited. Variations and combinations of the described features may be employed without departing from the scope of the invention as defined by the claims and their equivalents.

### Computing Environment, Security Controls, and Governed Data Handling

In certain embodiments, the clinician-bounded autopilot assistant is implemented using one or more computing systems comprising at least one processor, memory, and one or more communication interfaces. The computing systems execute instructions that implement telemetry ingestion, patient state estimation, policy evaluation, risk classification, admissibility validation, governance enforcement, secure execution, workflow orchestration, corrective action handling, and audit recording. The computing environment may be distributed across multiple physical or virtual components, provided that governance boundaries and safe failure behaviors are preserved.

In exemplary implementations, the system includes local or remote processing resources that receive physiologic monitoring telemetry and therapeutic-device telemetry, process such telemetry in near real time, and generate governed findings and candidate actions. Memory resources store executable instructions, policy artifacts, order sets, corridor constraints, device safety grammar profiles, patient-specific response profiles, and audit records. The system may further include local buffering and time-alignment mechanisms to accommodate variations in telemetry latency or network connectivity, while maintaining conservative actuation behavior when dependencies are degraded.

In certain embodiments, the system employs security controls to protect patient data, preserve integrity of governance artifacts, and prevent unauthorized actuation. Access to system functions may be restricted using authentication and role-based access control mechanisms, such that only authorized clinicians or system components may approve high-risk actions, modify order sets, adjust autonomy levels, or issue execution authorization artifacts. Communications between system components, devices, and clinical systems may be encrypted in transit, and sensitive records may be encrypted at rest in accordance with institutional policy and regulatory requirements.

In exemplary implementations, cryptographic mechanisms are used to protect execution authorization artifacts, audit records, and integrity checks. Execution tokens may be signed, hashed, or otherwise bound to an execution context to prevent forgery or reuse outside their permitted scope. In exemplary embodiments, governed records and execution authorization artifacts are not merely audit logs, but constitute runtime gating prerequisites such that progression of the closed-loop system to subsequent actions is prevented unless required compliance evidence is successfully generated and validated. Audit records may be protected using tamper-evident structures, such as hash-linked records or digital signatures, and the system may periodically verify integrity by recomputing hashes or validating signatures. These mechanisms support ledger-gated completion and post hoc verification without requiring any particular distributed ledger or blockchain technology.

In certain embodiments, the system enforces governance boundaries on data handling and retention. Governed data objects may include telemetry streams, patient state representations, candidate actions, authorization evidence, execution acknowledgements, corrective actions, and learning updates. Policies may define how long such data is retained, who may access it, and under what circumstances it may be exported or reviewed. In some embodiments, data minimization principles are applied such that only fields necessary for operational safety, auditability, and regulatory compliance are retained.

In exemplary implementations, the system supports generation of governed export artifacts for clinical review, compliance review, or incident investigation. Export artifacts may include selected audit records, governance explanations, and integrity proofs that allow a reviewer to verify that actions were taken within permitted corridors and under valid authorization. Export operations are governed and may be restricted by role-based access control to prevent inappropriate disclosure of sensitive information.

In certain embodiments, the system supports de-identification or pseudonymization of data for secondary use cases, such as performance evaluation or system improvement, while maintaining separation from operational decision-making. Such secondary artifacts are generated under governance control and do not affect real-time actuation or patient-specific control loops.

In exemplary implementations, the system adopts a fail-safe security posture. When security dependencies fail, such as when authentication cannot be confirmed, required cryptographic keys are unavailable, or integrity verification fails, the system defaults to a deny-by-default posture for actuation. In such cases, autonomous therapeutic-device control is suspended, autonomy levels may be downgraded, and escalation to clinician review occurs. Governed failure events are recorded to support transparency and remediation.

Accordingly, the computing environment, security controls, and data handling mechanisms described herein support safe and accountable operation of the clinician-bounded autopilot assistant. By integrating execution logic with governance enforcement, authorization control, and tamper-evident auditability, the system ensures that closed-loop assistance operates within regulated constraints and remains resilient to infrastructure failures, security threats, and operational variability.

### Governed Logging, Audit Records, and Explainability Outputs

In certain embodiments, the clinician-bounded autopilot assistant generates governed audit records and explainability outputs that support accountability, regulatory compliance, and post hoc review of closed-loop operation. Logging and audit are integral components of the execution path rather than ancillary monitoring functions, and are designed to link observation, interpretation, proposal, authorization, execution, and outcome into a traceable record suitable for regulated clinical environments.

In exemplary implementations, the system records telemetry-derived evidence, patient state representations, candidate therapeutic-device control commands, admissibility determinations, risk classifications, authorization evidence, execution authorization artifacts, dispatch events, device acknowledgements, post-dispatch observations, conformance assessments, and any corrective actions executed. These records are associated with identifiers for the patient encounter, therapeutic device, order set version, corridor constraints, autonomy level, and applicable policy artifacts. By associating each action with its governing context, the system enables reconstruction of why an action was permitted, denied, escalated, or corrected.

In certain embodiments, audit records are written to a tamper-evident audit store implemented as an append-only record sequence protected by cryptographic integrity mechanisms. Records may be hash-linked, digitally signed, or otherwise protected such that post hoc modification is detectable. The system may treat successful append of a required audit record as a prerequisite for recognizing action completion, consistent with ledger-gated completion rules. When required records cannot be written, the system withholds completion acknowledgment and transitions to safe behavior.

In exemplary implementations, audit records include provenance metadata identifying telemetry sources, interface identifiers, timestamps, and signal-quality or confidence indicators. When derived indices or model-based interpretations are used, the records may further include references to the computational method, confidence outputs, and any assumptions or confirmation behaviors applied. This provenance information enables later evaluation of evidence quality and supports defensible review of system behavior.

In certain embodiments, the system generates governance explanations that summarize the rationale for permitting or denying an action. Governance explanations may identify the corridor constraints applied, the autonomy level in effect, the policy rules evaluated, the admissibility determination, and any authorization evidence required or satisfied. These explanations may be generated prior to execution, upon denial or escalation, or upon request by an authorized clinician. The explanations are recorded as governed artifacts and may be presented in a human-readable form without exposing proprietary policy internals.

In exemplary implementations, explainability outputs are structured to distinguish observed evidence from inferred conclusions and to indicate associated confidence levels. When actions are withheld due to uncertainty or insufficient evidence, the system records the basis for the decision and any confirmation tasks requested. This approach supports transparency and reduces the risk that automated assistance is perceived as opaque or arbitrary.

In certain embodiments, the system supports generation of export artifacts for clinical review, quality assurance, compliance audits, or incident investigation. Export artifacts may include selected audit records, governance explanations, and integrity proofs that demonstrate record completeness and tamper-evidence. Export operations are governed by access control policies and may provide role-specific views, such that clinicians, compliance officers, and technical reviewers receive information appropriate to their responsibilities.

In exemplary implementations, audit records and explainability outputs are retained and managed according to governance policies that define retention duration, access permissions, and conditions for deletion or archival. In some embodiments, the system supports de-identification or pseudonymization of audit artifacts for secondary analysis or system improvement, while ensuring that such artifacts are not used for real-time actuation or to bypass governance controls.

Accordingly, the governed logging, audit, and explainability mechanisms described herein ensure that the clinician-bounded autopilot assistant operates transparently and accountably. By embedding audit generation and explanation into the execution path, the system provides verifiable evidence that actions were taken, or withheld, within clinician-defined corridors and governance constraints, thereby supporting regulated deployment, clinical trust, and legal defensibility.

### Multi-Patient Operation, Fleet-Level Governance, and Institutional Control

In certain embodiments, the clinician-bounded autopilot assistant supports operation across multiple patients, devices, and clinical units while preserving patient-specific control, clinician authority, and institutional governance. Multi-patient operation is implemented in a report-first and supervision-oriented posture, such that bounded autonomous actuation remains scoped to individual patients under active clinician-authored order sets, while higher-level monitoring and governance functions support clinician situational awareness and institutional oversight.

In exemplary implementations, any multi-patient views, queues, summaries, or institutional analytics are derived from patient-specific governed findings and audit records produced by the canonical loop and do not create independent actuation authority or a separate control loop. Any bounded actuation remains patient-scoped and order-set-scoped, and is executed only through the same validation, authorization, and ledger-gated completion path.

In exemplary implementations, the system provides a multi-patient monitoring view that summarizes governed patient states, stability indicators, and risk classifications derived from patient-specific closed-loop operation. The summarized view may indicate which patients are operating within corridor constraints, which patients are approaching or exceeding corridor bounds, and which patients require clinician attention due to uncertainty, escalation, or suspended autonomy. These summaries are derived from governed findings and audit records and do not bypass patient-specific authorization requirements.

In certain embodiments, the system supports institutional management of order sets, corridor templates, safety grammar profiles, and governance policies as versioned artifacts. Such artifacts may be maintained under institutional change control workflows that include review, approval, effective dates, and rollback capability. When a policy or order set is updated, the system associates each version with a unique identifier and records which version was active at the time of each governed action, thereby preserving traceability and accountability.

In exemplary implementations, fleet-level updates are deployed using staged or controlled rollout mechanisms. Updates may be applied to a limited subset of patients, devices, or clinical units prior to broader deployment, and governed outcomes may be monitored to detect unintended effects such as increased escalation frequency, deviation rates, or corrective action usage. When adverse patterns are detected, the system may automatically downgrade autonomy, suspend actuation under the updated policy, or revert to a prior version, while recording governed events documenting the basis for the change.

In certain embodiments, the system distinguishes between patient-specific artifacts and institutional artifacts. Patient-specific artifacts, such as response profiles or encounter-specific audit records, are maintained separately from institutional artifacts, such as corridor templates and safety grammar profiles. Updates to patient-specific artifacts are governed and auditable on a per-patient basis, while updates to institutional artifacts follow institutional governance workflows. This separation prevents unintended propagation of patient-specific behavior across the fleet.

In exemplary implementations, the system supports comparative analysis across patients and deployments to assist institutional governance and quality improvement. Such analysis may include aggregation of governed metrics such as escalation frequency, persistence violations, corrective action invocation, or authorization delays. Aggregated analysis is performed in a manner that preserves patient privacy and does not affect real-time actuation or closed-loop decision-making for individual patients.

In certain embodiments, institutional governance includes the ability to suspend or restrict autopilot operation across a set of devices or clinical units in response to policy changes, safety alerts, or detected anomalies. Such actions may include downgrading autonomy levels, requiring additional authorization evidence, or enforcing report-only operation until review is completed. These institutional controls are themselves governed actions and are recorded with policy identifiers and timestamps.

In exemplary implementations, the system supports clinician supervision across multiple patients by prioritizing attention to patients exhibiting sustained deviation, uncertainty, or repeated escalation. The system may surface governance explanations and audit summaries for selected patients to assist clinicians in rapid review and intervention. These supervisory features enhance clinician situational awareness without replacing patient-specific clinical decision-making.

Accordingly, multi-patient operation and fleet-level governance are implemented in a manner that scales clinician supervision while preserving clinician-bounded control and auditability. By separating patient-specific closed-loop assistance from institutional policy management and by enforcing governance at both levels, the system supports safe deployment across complex clinical environments with multiple patients, devices, and evolving policies.

### Canonical Governed Closed-Loop Operation of the Clinician-Bounded Autopilot Assistant

This section describes a canonical end-to-end operation of the clinician-bounded autopilot assistant to which all embodiments, variations, and clinical profiles described in this specification conform. The purpose of this walkthrough is to illustrate how telemetry ingestion, interpretation, governance enforcement, authorization, execution, observation, and escalation operate together as a single governed closed loop. The described operation is exemplary and non-limiting and is intended to demonstrate system behavior rather than to define separate inventions or control loops.

Clinical Profiles as Non-Limiting Parameterizations. In this specification, any “clinical profile” (e.g., infusion titration, ventilator titration, capnography-gated evidence quality, arterial-line-derived indices) is a non-limiting parameterization of the same canonical governed closed loop described herein. Profiles differ only in (i) which evidence sources are emphasized, (ii) which governed action classes are enabled by the autonomy level, and (iii) which corridor constraints and device-grammar rules apply. A profile does not constitute a distinct system, a distinct control loop, or a separate invention.

In exemplary implementations, operation begins when an authorized clinician activates or selects an order set for a patient encounter. The order set defines clinical intent by specifying corridor constraints, high-risk criteria, permitted action classes, and an autonomy level. Upon activation, the system binds an execution context that associates the patient identity, therapeutic-device identities, corridor identifiers, order set version identifiers, and applicable policy identifiers. This execution context governs all subsequent closed-loop activity until modified or terminated by clinician input or policy.

The system continuously ingests physiologic monitoring telemetry and therapeutic-device telemetry associated with the patient. Telemetry is processed to generate a patient state representation that includes physiologic state variables, device status indicators, timestamps, and confidence or signal-quality indicators. The system interprets telemetry under governance control, applying artifact detection, source reconciliation, and persistence confirmation to distinguish transient fluctuations from sustained conditions. When evidence sufficiency is not satisfied, the system refrains from actuation and initiates confirmation behavior or escalation rather than proceeding.

Based on the patient state representation and the active corridor constraints, the system generates governed findings and evaluates whether the patient state is within bounds, trending toward a bound, or projected to violate a bound. When bounded action is permitted by the autonomy level and evidence sufficiency is satisfied, the system proposes one or more candidate therapeutic-device control commands or other governed actions, such as monitoring cadence adjustments. Proposed actions are constrained by corridor limits, device capabilities, and transition constraints.

Prior to any execution, the system validates each candidate action under governance control. Validation includes admissibility determination using a device-specific safety grammar, risk classification based on projected outcomes and order set criteria, and evaluation of authorization requirements. When a candidate action is classified as high-risk or otherwise requires authorization, the system requests and verifies authorization evidence from an authorized clinician. When any required condition is not satisfied, execution is withheld and a governance explanation is generated.

When a candidate action is permitted, the system issues or validates an execution authorization artifact bound to the execution context and dispatches the action through a validation and execution interface. The system monitors device acknowledgements and associates the dispatch with a post-execution observation window. Completion of the action is recognized only when required governed records are successfully created and appended to a tamper-evident audit store in accordance with ledger-gated completion rules.

Following execution, the system observes post-dispatch telemetry to assess patient response and device behavior relative to corridor constraints. Persistence criteria are applied to determine whether the response reflects convergence, overshoot, oscillation, non-response, or sustained deviation. When the response indicates stability, the closed loop continues monitoring within the current corridor. When a sustained deviation or non-convergence is detected, the system invokes corrective-action logic under governance control, selecting and executing an appropriate corrective procedure or escalating to clinician review as required.

Throughout operation, the system maintains a patient-specific response profile that characterizes how the patient responds to bounded actions over time. Updates to the response profile are performed only using governed episodes that satisfy evidence sufficiency criteria and are constrained by policy to prevent uncontrolled drift. Learning updates are recorded with policy identifiers and order set versions to preserve auditability and accountability.

In some embodiments, the patient-specific response profile is optional and may be omitted or replaced by fixed protocol rules, clinician-authored heuristics, or non-learning models. When used, the response profile is applied only to the response projection step to improve estimation under governance, and does not expand permitted action classes, corridor bounds, or authorization requirements.

Human interaction is treated as an explicit control input in the closed loop. Clinician responses to recommendations, confirmation requests, authorization prompts, or escalation notifications may modify corridor constraints, autonomy levels, or permitted action classes. Such modifications update the execution context and govern subsequent closed-loop iterations. When a clinician pauses, overrides, or terminates autopilot operation, the system immediately suspends further actuation and records the event under governance control.

The closed-loop operation continues until a termination condition is met. Termination conditions may include sustained achievement of stability criteria, discontinuation of the active order set, clinician override or pause, policy-mandated suspension, or completion of a defined clinical objective. Upon termination, the system records the basis for termination and preserves the audit trace of the closed-loop operation for review.

Accordingly, the clinician-bounded autopilot assistant operates as a governed closed-loop system that listens, interprets, proposes, validates, executes, observes, adapts, and escalates under explicit clinician-defined constraints and institutional policy. All embodiments described in this specification implement variations of this canonical operation, and no embodiment departs from the fundamental posture of clinician-bounded authority, governance enforcement, and auditability suitable for regulated clinical environments.

### Embodiment Profiles of the Canonical Governed Closed-Loop Operation (Non-Limiting)

In exemplary implementations, the canonical governed closed-loop operation described in previous section may be instantiated in multiple embodiment profiles. Each profile is a non-limiting parameterization of the same canonical governed loop—and not a distinct control system—and differs primarily in (i) which evidence sources are emphasized during the Observe and Interpret stages, (ii) which therapeutic-device command classes are enabled under the active autonomy level, and (iii) which corridor constraints, admissibility rules, and authorization conditions apply.

Across all profiles, the same canonical loop is preserved, including: observing physiologic monitoring signals and therapeutic-device telemetry; interpreting such telemetry into governed findings with associated confidence indicators; evaluating corridor constraints and autonomy permissions; selecting a report-only, recommendation, or bounded actuation posture; validating admissibility and gating execution; authorizing high-risk actions as required; dispatching permitted commands; observing post-dispatch response and detecting non-convergence or sustained deviation; escalating or revising as needed; and recording governed audit artifacts. Differences among profiles do not alter this canonical loop, but instead reflect variations in evidence emphasis and permitted actuation within clinician-defined bounds.

### Profile A—Infusion Titration Within Bounded Corridors

In this profile, the Observe stage emphasizes ingestion of physiologic monitoring signals, vasoactive infusion telemetry, and infusion status indicators such as rate, reservoir volume, occlusion alarms, or depletion forecasts. The Interpret stage produces governed findings reflecting current and trending physiologic and device conditions, with associated confidence indicators. The Corridor and Autonomy stage enforces clinician-authored infusion corridors, step-size limits, rate-of-change constraints, and adjustment cadence permissions derived from the active order set.

The Bounded Actuation stage applies incremental infusion-rate adjustments only within permitted corridors and autonomy levels. When response behavior fails to converge, oscillates, or violates persistence criteria, the system invokes escalation and governed query behavior as described elsewhere in this specification. Clinician responses, such as modifying targets or corridor bounds, are treated as governed control inputs that update the permitted action space for subsequent closed-loop iterations.

### Profile B—Ventilator Titration With Coupled Constraints

In this profile, the Observe stage emphasizes ventilator telemetry, including pressure, volume, and flow signals, together with gas-exchange evidence and relevant physiologic monitoring signals. The Interpret stage generates governed findings describing ventilatory and respiratory state within the confines of the canonical loop. The Corridor and Autonomy stage enforces clinician-defined ventilator corridors and coupled parameter constraints that restrict how multiple ventilator settings may be adjusted together.

The Bounded Actuation stage applies ventilator setting changes only when admissibility validation under device-specific safety grammar rules is satisfied and when permitted by the active autonomy level. When the system cannot maintain corridor conformance, encounters ambiguous telemetry, or detects elevated risk, it downgrades autonomy, suppresses actuation, and escalates to clinician review in accordance with governance and authorization rules, without departing from the canonical loop.

### Profile C—Capnography as Evidence-Quality Gating

In this profile, capnography waveforms and derived indicators are used primarily as evidence-quality and gating inputs within the Interpret and Validate stages. Capnography-derived governed findings influence whether downstream actuation is permitted, must be limited to report-only or recommendation modes, or must be escalated to clinician review. Any waveform interpretation, classification, or feature extraction described in this specification is an optional implementation detail and is not required to practice the canonical loop.

Capnography in this profile does not serve as a mandatory control variable. Rather, it refines evidence sufficiency determination, risk classification, and confirmation behavior. Absence of capnography does not prevent operation of the canonical governed loop, which continues using whatever authorized evidence sources are available.

### Profile D—Arterial-Line Indices as Optional Derived Evidence

In this profile, arterial pressure waveform-derived indices, when available, are incorporated as optional governed evidence inputs to the Interpret and Validate stages. Using published and well-known techniques for enablement, the system may derive indices reflecting hemodynamic behavior and incorporate such indices into governed findings with associated confidence indicators and provenance metadata.

The canonical loop does not require the presence of an arterial line. When such monitoring is unavailable, the system continues operating using other authorized telemetry sources under the same governance, persistence, and escalation logic. When present, arterial line-derived indices refine evidence assessment and prioritization of actions, without altering the fundamental clinician-bounded control posture.

### Definitions

Clinician-Bounded—refers to operation of a computing system in which autonomous or semi-autonomous control actions affecting a therapeutic device are constrained by parameters, permissions, limits, or conditions expressly defined or authorized by a clinician through a clinician-authored order set or equivalent clinical directive. A clinician-bounded system is not permitted to operate outside such bounds without clinician involvement, authorization, or intervention, and does not replace clinical judgment, diagnosis, or treatment selection.

Therapeutic Device—refers to a medical device, system, or apparatus configured to deliver, regulate, modify, or influence therapy, treatment, or physiologic support to a patient through physical operation. A therapeutic device includes devices capable of receiving control commands that affect device configuration, parameters, mode, or behavior, and may include infusion systems, ventilators, dialysis machines, or other regulated medical devices.

Therapeutic-Device Control Command—refers to a machine-readable instruction or directive expressed in a form suitable for controlling operation of a therapeutic device, including adjusting configuration parameters, changing operational modes, initiating or suspending device functions, or otherwise modifying device behavior. A therapeutic-device control command affects physical operation of the therapeutic device and is distinct from a recommendation, alert, or advisory output that does not itself cause device actuation.

Physiologic Monitoring Signal—refers to a machine-readable measurement, data stream, or derived value representing a physiologic parameter of a patient, obtained from one or more sensors, monitoring devices, or clinical systems. Physiologic monitoring signals may be continuous or intermittent and may include raw measurements, processed values, trends, timestamps, or associated signal-quality indicators suitable for computational evaluation.

Device Telemetry—refers to machine-readable data describing operational state, performance, configuration, status, or outputs of a therapeutic device. Device telemetry may include parameter settings, alarms, acknowledgements, operational modes, execution feedback, timestamps, or other data generated by the therapeutic device before, during, or after execution of a therapeutic-device control command.

Patient State Representation—refers to a machine-readable representation of patient-related context derived from one or more physiologic monitoring signals, device telemetry, or associated clinical data, and structured for use in computational evaluation or control. A patient state representation reflects current physiologic and device conditions relevant to therapeutic-device control and does not imply clinical diagnosis, treatment selection, or medical decision-making reserved to a clinician.

Functionally Equivalent Computed State Abstraction or Computed State Abstraction—refers to a computed representation that, while not explicitly stored or labeled as a patient state representation, provides equivalent informational content sufficient to support evaluation, control, or decision logic associated with therapeutic-device control. A functionally equivalent computed state abstraction may be derived on demand, transiently computed, or distributed across processing components, provided it reflects current physiologic and device conditions used to govern system behavior. For purposes of this disclosure, equivalence refers to the ability of the computed state abstraction to support the same therapeutic-device control command proposal, validation, dispatch eligibility, and conformance monitoring decisions as a patient state representation within the claimed system.

Autonomy Level—refers to a machine-interpretable designation obtained from a clinician-authored order set or equivalent clinical directive that defines which classes of system actions are permitted to occur autonomously, which actions require additional authorization or confirmation, and which actions are prohibited. An autonomy level constrains system behavior at runtime and governs whether therapeutic-device actuation may occur without further clinician involvement.

Corridor Constraint—refers to a bounded range, threshold, or equivalent policy-defined limit that constrains permissible values, transitions, or operating regions for one or more physiologic state variables or therapeutic-device parameters. Corridor constraints define acceptable operating boundaries for closed-loop control and are used for execution constraint enforcement rather than for clinical diagnosis or treatment determination.

Sustained Deviation—refers to a condition in which observed physiologic monitoring signals or device telemetry diverge from one or more corridor constraints beyond a transient excursion, as determined using one or more persistence criteria. Persistence criteria may include a defined duration, a threshold number of consecutive samples, or a sliding time window satisfying a nonconformance condition, such that brief artifacts or short-lived fluctuations do not trigger corrective action.

Corrective Action—refers to an action initiated by a computing system in response to detection of a sustained deviation from one or more corridor constraints and intended to restore operation of a therapeutic device toward a permitted, safer, or more stable state. A corrective action may include rollback to a prior known-safe configuration, compensatory adjustment of one or more parameters, transition to a device-specific safe mode, suspension of autonomous actuation, or other actions affecting operation of the therapeutic device without implying clinical diagnosis or treatment selection.

Expected Response—refers to a predicted, projected, or reference outcome associated with execution of a therapeutic-device control command, derived from one or more patient state representations, corridor constraints, device models, prior observed responses, or policy-defined limits. An expected response is used for comparison with observed post-execution telemetry to assess conformance or deviation and does not represent a guaranteed clinical outcome.

Deviation Metric—refers to a quantitative or qualitative measure representing divergence between an observed physiologic or device response and an expected response or corridor constraint. A deviation metric may be computed using differences, thresholds, trend analysis, probability estimates, or other evaluation techniques suitable for determining whether system behavior remains within permitted bounds.

Deviation Event or Deviation Condition—refers to a detected condition in which a deviation metric exceeds a defined tolerance or satisfies a nonconformance criterion relative to one or more corridor constraints. A deviation event may be transient or sustained and serves as an input to persistence evaluation, corrective-action selection, or governance enforcement.

Constraint Safety Grammar or Constraint and Safety Grammar—refers to a formally defined, machine-enforceable specification that defines and constrains permissible command structures, parameter values, and parameter transitions for therapeutic-device control commands. A constraint safety grammar or constraint and safety grammar is used to validate candidate therapeutic-device control commands prior to execution to ensure compliance with semantic bounds, transition constraints, coupled constraints, and device-specific safety rules, and operates independent of clinical intent, diagnosis, or treatment selection. By way of non-limiting example, a constraint safety grammar may specify that a therapeutic-device parameter adjustment shall not exceed a defined step-size increment per control iteration, that cumulative adjustments shall not exceed a maximum rate-of-change over a defined time window, or that certain parameter transitions are prohibited unless prerequisite device states or coupled parameter conditions are satisfied.

Coupled Constraint—refers to a constraint that restricts permissible values or transitions of one therapeutic-device parameter based on the value, state, or transition of one or more other parameters. A coupled constraint enforces inter-parameter safety relationships and prevents execution of control commands that would otherwise satisfy individual parameter limits but violate combined safety conditions.

Step-Size Constraint—refers to a constraint that limits the magnitude of change applied to a therapeutic-device parameter between successive control actions. A step-size constraint is used to prevent abrupt or excessive adjustments that could compromise patient safety or device stability.

Rate-of-Change Constraint—refers to a constraint that limits the temporal rate at which a therapeutic-device parameter may change as a result of one or more control actions. A rate-of-change constraint restricts how quickly a parameter may be adjusted over time, independent of absolute parameter bounds.

Command Rejection—refers to a determination by a computing system that a candidate therapeutic-device control command is not permitted to proceed toward execution due to violation of one or more corridor constraints, constraint safety grammar rules, authorization requirements, or governance conditions. Command rejection prevents the rejected command from affecting physical operation of the therapeutic device.

Response Evaluation—refers to a computational process that compares post-execution telemetry received from a therapeutic device and associated physiologic monitoring signals to an expected response, corridor constraint, or deviation metric in order to determine conformance, transient deviation, or sustained deviation.

Governance Engine—refers to a computing component configured to evaluate proposed therapeutic-device control actions against one or more machine-interpretable constraints, policies, or rules and to determine how such actions are permitted to proceed. A governance engine may classify a proposed action as permitted, modified, blocked, escalated, or subject to additional conditions, without itself directly executing the action on a therapeutic device.

Governance Enforcement Engine—refers to a computing component configured to enforce governance outcomes by technically preventing or allowing progression of a therapeutic-device control action toward execution or completion unless specified compliance requirements are satisfied. Governance enforcement is performed through deterministic system logic and does not rely solely on procedural compliance, manual review, or post-hoc auditing.

Execution Path—refers to the sequence of system operations, components, and interfaces through which a proposed therapeutic-device control command progresses from proposal through validation, authorization, dispatch, and completion. The execution path may include governance evaluation, execution gating, record creation, device interfacing, and receipt of execution acknowledgements.

Execution Gating—refers to a technical mechanism by which progression along an execution path is prevented, delayed, or conditioned unless one or more predefined requirements are satisfied. Execution gating may be applied prior to dispatch, prior to execution completion, or between stages of the execution path, and may be based on authorization, validation, governance, or record-creation conditions.

Execution Completion—refers to a system-recognized state in which a therapeutic-device control command is treated as finalized or effective for purposes of closed-loop progression. Execution completion expressly excludes intermediate or preparatory stages that remain subject to execution gating, validation, authorization, or governance enforcement.

Governed Record—refers to a machine-readable record generated by a computing system in association with a therapeutic-device control action, the record encoding execution-relevant information such as execution context, applied constraints or policies, authorization status, and execution state. A governed record may be used as a condition for execution gating, execution completion, or auditability.

Governed Record Store—refers to a non-transitory data storage system configured to store governed records in a manner that supports integrity verification and controlled access. A governed record store may participate as an active component in execution gating or governance enforcement, rather than serving solely as a passive audit repository.

Tamper-Evident—refers to a property of a data storage system or record in which unauthorized modification, deletion, or reordering of stored data is detectable through integrity verification mechanisms. Tamper-evident storage does not require prevention of all modification attempts but enables detection of unauthorized changes.

Ledger-Gated Completion—refers to a form of execution gating in which execution completion of a therapeutic-device control command is prevented unless a compliant governed record associated with the command is successfully created and stored in a governed record store. Ledger-gated completion is an optional embodiment of governance enforcement and is not required for all implementations.

Execution Authorization Artifact or Execution Scope—refers to a machine-readable artifact, representation, or encoded specification generated by a computing system that defines a permitted set of conditions under which a therapeutic-device control command is authorized to be dispatched or treated as completed. The execution authorization artifact or execution scope may specify one or more constraints including patient identity, therapeutic-device identity, action class, corridor identifier, permitted parameter ranges, order set version, or allowable time window, and may be evaluated by a validation and execution interface to verify that the therapeutic-device control command is authorized prior to dispatch or execution completion. In exemplary embodiments, an execution authorization artifact encodes a bounded execution scope including at least a patient identifier, a therapeutic-device identifier, an authorized action class, an applicable corridor identifier, one or more permitted parameter ranges or transition constraints, and an allowable time window during which the action may be dispatched.

Authorization Engine—refers to a computing component configured to evaluate whether a proposed therapeutic-device control command satisfies one or more authorization requirements defined by a clinician-authored order set, institutional policy, or system configuration. The authorization engine determines whether execution of the proposed command is permitted, escalated, or prohibited, without itself performing device control.

Authorization Requirement—refers to a machine-interpretable condition that must be satisfied before a therapeutic-device control command is permitted to proceed toward dispatch or execution completion. An authorization requirement may depend on clinician role, confirmation input, autonomy level, action classification, or other policy-defined criteria.

Authorization Result—refers to a machine-readable outcome indicating whether an authorization requirement applicable to a therapeutic-device control command has been satisfied. An authorization result may indicate approval, denial, escalation, or pending status and may be used as an input to execution gating or governance enforcement.

Authenticated Role—refers to an identity, credential, or role that has been verified by a computing system and is associated with defined authorization privileges. An authenticated role may correspond to a clinician, system process, or authorized interface permitted to satisfy an authorization requirement.

Multi-Party Authorization—refers to an authorization requirement in which approval from two or more authenticated roles or entities is required before a therapeutic-device control command is permitted to proceed. Multi-party authorization may be applied selectively based on risk classification, action type, or corridor constraints.

Validation and Execution Interface—refers to a system interface configured to receive validated and authorized therapeutic-device control commands and to cause dispatch of such commands to a therapeutic device, directly or via an intermediary execution component. The validation and execution interface enforces execution gating and receives execution acknowledgements or post-dispatch telemetry.

Acknowledgement or Execution Acknowledgement—refers to a machine-readable indication received from a therapeutic device or execution interface confirming receipt, acceptance, or completion of a dispatched therapeutic-device control command. An acknowledgement may be used to determine execution completion or to trigger corrective or escalation behavior.

Post-Dispatch Telemetry—refers to machine-readable data received after dispatch of a therapeutic-device control command that describes resulting device state, physiologic measurements, execution outcomes, or error conditions. Post-dispatch telemetry is used to evaluate conformance to corridor constraints and to detect sustained deviations.

Omission Event—refers to a system-detected condition in which a required action, confirmation, reassessment, escalation, or bounded adjustment has not occurred within a policy-defined or order-set-defined context. An omission event is identified based on absence of a required action or state transition and is not limited to expiration of a fixed timer.

Omission Correction—refers to a proposed or initiated action intended to remediate, compensate for, or otherwise address an omission event. An omission correction may include escalation to clinician review, suspension of autonomous actuation, generation of a confirmation request, or initiation of a corrective action through the governed execution path.

Patient State Vector—refers to a multi-dimensional instance of a patient state representation comprising a set of values, parameters, or features corresponding to physiologic variables, device-associated variables, or derived indicators. A patient state vector is one possible implementation of a patient state representation and is not required to have a fixed dimensionality or specific mathematical form.

State Estimation Module—refers to a computing component configured to derive a patient state representation from physiologic monitoring signals and therapeutic-device telemetry. State estimation may include filtering, aggregation, normalization, confidence assessment, or other computational processes and does not require predictive modeling or clinical inference.

State Snapshot—refers to a representation of a patient state representation corresponding to a particular point in time or time window. A state snapshot may be used for comparison, persistence evaluation, audit association, or corrective-action selection.

Physiologic Corridor—refers to a multi-dimensional set of corridor constraints defining an acceptable operating region for patient-related variables and/or device-influenced parameters. A physiologic corridor is used to constrain autonomous or semi-autonomous device control actions and does not constitute a medical diagnosis or treatment recommendation.

Persistence Criterion—refers to a rule or condition that specifies when a deviation relative to a corridor constraint is considered sustained rather than transient. A persistence criterion may be defined using a duration, a number of consecutive samples, a sliding time window, or an equivalent temporal or contextual condition.

Safe-Mode Procedure—refers to a predefined corrective action that transitions a therapeutic device to a configuration or operating mode designated as safer or more conservative under applicable policy or device constraints. A safe-mode procedure is executed through the same validation, authorization, and execution controls as other therapeutic-device control commands.

Command Proposal Module—refers to a computing component configured to generate one or more candidate therapeutic-device control commands based on a patient state representation and applicable corridor constraints when therapeutic-device actuation is permitted by an autonomy level.

Candidate Therapeutic-Device Control Command—refers to a proposed therapeutic-device control command that has been generated but has not yet been validated, authorized, or dispatched for execution. A candidate therapeutic-device control command is subject to evaluation under corridor constraints, authorization requirements, and other execution conditions.

Response Projection—refers to a computational process that estimates an expected physiologic or device response associated with execution of a candidate therapeutic-device control command, based on patient state representation, corridor constraints, historical response data, device characteristics, or combinations thereof.

Projected Outcome Metric—refers to a quantitative or qualitative value generated by response projection that represents an expected effect or consequence of executing a candidate therapeutic-device control command, and that may be evaluated against a risk criterion prior to dispatch.

Risk Criterion—refers to a machine-interpretable condition defined by a clinician-authored order set or applicable policy that specifies whether a projected outcome metric satisfies an acceptable level of risk for execution of a therapeutic-device control command.

Evidence Sufficiency Criterion—refers to a machine-interpretable condition that specifies whether available physiologic monitoring signals, device telemetry, confidence indicators, or signal-quality indicators are sufficient to permit proposal or dispatch of a therapeutic-device control command.

Authorization Enforcement Module—refers to a computing component configured to prevent dispatch of a therapeutic-device control command unless applicable authorization requirements are satisfied.

Grammar Validation—refers to evaluation of a candidate therapeutic-device control command against a constraint and safety grammar to determine whether the command is admissible for execution.

Admissibility—refers to a determination that a candidate therapeutic-device control command satisfies applicable grammar rules, corridor constraints, and authorization requirements necessary to permit dispatch.

Conformance Monitoring—refers to a computational process by which post-dispatch telemetry is evaluated relative to corridor constraints to determine whether system behavior remains within permitted bounds over time.

Transient Excursion—refers to a temporary departure from a corridor constraint that does not satisfy criteria for sustained deviation and does not, by itself, trigger corrective action.

Rollback Procedure—refers to a corrective action that restores a therapeutic device to a prior known-safe configuration or operational state following detection of a sustained deviation.

Suspension of Autonomous Actuation—refers to a system-enforced transition in which autonomous therapeutic-device control is halted and the system operates in a report-only, advisory, or clinician-review mode until specified prerequisites for autonomous actuation are satisfied.

Governed Record—refers to a machine-readable record generated by a computing system that documents execution-relevant information associated with a therapeutic-device control command, including at least a policy basis under which the command is permitted and any required authorization evidence.

Compliant Governed Record—refers to a governed record that satisfies one or more structural, content, or integrity requirements defined by applicable policy and is therefore eligible to be used as a condition for execution progression or completion recognition.

Execution Progression—refers to advancement of a closed-loop control process from one control iteration to a subsequent iteration, including recognition that a previously dispatched therapeutic-device control command has completed for purposes of enabling further autonomous actuation.

Execution Scope—refers to a bounded set of conditions under which a therapeutic-device control command is permitted to be dispatched, including one or more of patient identity, therapeutic-device identity, action class, corridor identifier, permitted parameter ranges, order set version, or allowable time window.

Authorization Evidence—refers to machine-readable data indicating satisfaction of an authorization requirement, including confirmations, approvals, acknowledgements, or cryptographically verifiable credentials associated with an authenticated role.

Tamper-Evident Audit Store—refers to a non-transitory data storage system configured such that unauthorized modification or deletion of stored records is detectable through integrity verification mechanisms, including logical or cryptographic linkage between records.

Execution Failure—refers to a system-recognized condition in which a therapeutic-device control command is treated as not completed due to failure to satisfy governance, authorization, validation, or record-creation requirements, thereby inhibiting further autonomous progression of the closed-loop control system.

Confidence or Signal-Quality Indicator—refers to machine-readable information associated with physiologic monitoring signals or device telemetry that reflects reliability, accuracy, timeliness, or integrity of the data.

Governed Confirmation Request—refers to a system-generated request for additional information, action, or acknowledgment that must be satisfied before autonomous therapeutic-device actuation may proceed, including requests for additional measurements, increased monitoring cadence, or clinician confirmation.

Unresolved System State—refers to a control state in which one or more governed confirmation requests remain unsatisfied, thereby preventing further autonomous therapeutic-device actuation until resolution in accordance with a clinician-authored order set or applicable policy.

Monitoring Cadence Controller—refers to a computing component configured to modify the frequency or timing of physiologic monitoring or measurement based on corridor proximity, predicted outcomes, evidence sufficiency, or detected deviation conditions.

Measurement Cadence—refers to the temporal frequency at which physiologic monitoring signals are acquired, sampled, or updated.

Coupled Transition Constraint—refers to a constraint that restricts permissible changes to one therapeutic-device parameter based on the value or state of one or more other therapeutic-device parameters, such that multi-parameter safety relationships are enforced prior to execution.

Clinical Profile—a configuration of the canonical governed closed loop that specifies which evidence sources, action classes, corridor constraints, and device-specific grammar rules apply, without altering the canonical sequence of governed stages.

Governed Action—any system action selected within the canonical loop, including reporting, recommendation/query, monitoring cadence adjustment, or therapeutic-device control command dispatch, that is subject to governance validation, authorization (when required), and audit recording.

### DETAILED DESCRIPTION OF THE FIGURES

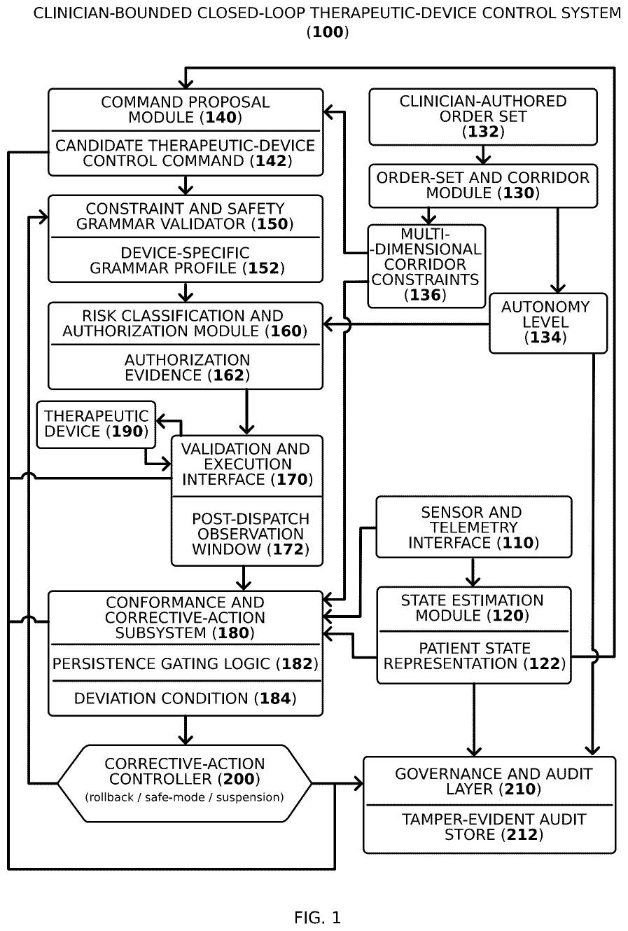

### FIG. 1

FIG. 1 illustrates an exemplary embodiment of a clinician-bounded, policy-governed closed-loop therapeutic-device control system (100) for use in a regulated clinical environment. The system (100) is configured to issue and enforce therapeutic-device control commands that affect physical operation of at least one therapeutic device under clinician-defined boundaries, governance constraints, authorization rules, persistence criteria, and corrective-action safeguards.

The governance enforcement engine, authorization gating, and admissibility grammar cooperate as a single integrated runtime execution path that conditions whether and how therapeutic-device control commands are dispatched and completed, thereby preventing unsafe, unauthorized, or silent therapeutic-device actuation.

The system (100) includes a sensor and telemetry interface (110) configured to receive real-time physiologic monitoring signals associated with a patient and therapeutic-device telemetry associated with one or more therapeutic devices (190). The received telemetry includes timestamps, source identifiers, sampling cadence information, and signal-quality or confidence indicators. The sensor and telemetry interface (110) normalizes incoming signals from heterogeneous sources into governed telemetry streams suitable for downstream processing.

Telemetry from the sensor and telemetry interface (110) is provided to a state estimation module (120) configured to generate a patient state representation (122). The patient state representation (122) includes one or more physiologic state variables, therapeutic-device status variables, temporal context, and at least one confidence or signal-quality indicator. The confidence or signal-quality indicator is used to control whether therapeutic-device control commands may be proposed, executed, or withheld pending confirmation or escalation.

The system (100) further includes an order-set and corridor module (130) configured to obtain an active clinician-authored order set (132) defining therapeutic intent and governance constraints. The clinician-authored order set (132) specifies an autonomy level (134) defining permitted and prohibited classes of system action, including whether autonomous therapeutic-device actuation is allowed, conditionally allowed, or prohibited. The order-set and corridor module (130) further defines one or more multi-dimensional corridor constraints (136) that establish permitted bounds for physiologic state variables and/or therapeutic-device parameters, thereby defining a bounded action space for closed-loop operation.

When autonomous actuation is permitted by the autonomy level (134), a command proposal module (140) evaluates the patient state representation (122) relative to the corridor constraints (136) and generates one or more candidate therapeutic-device control commands (142). The candidate therapeutic-device control commands (142) represent bounded, incremental adjustments constrained by clinician-defined step-size and rate-of-change limits and are expressed independently of device-specific command syntax.

Each candidate therapeutic-device control command (142) is evaluated by a constraint and safety grammar validator (150) configured to determine admissibility using a device-specific grammar profile (152). The device-specific grammar profile (152) defines permitted command forms, semantic parameter bounds, and prohibited parameter transitions, including step-size limits, rate-of-change limits, and coupled parameter constraints. When a candidate therapeutic-device control command (142) violates an admissibility constraint, the constraint and safety grammar validator (150) rejects the command or produces an admissibility failure reason identifying the violated constraint.

The system (100) further includes a risk classification and authorization module (160) configured to classify each admissible candidate therapeutic-device control command (142) as low-risk or high-risk based on projected outcomes, clinician-defined thresholds, and policy criteria specified by the clinician-authored order set (132). When a candidate therapeutic-device control command (142) is classified as high-risk, the risk classification and authorization module (160) prohibits dispatch absent authorization evidence (162) from an authorized clinician. The authorization evidence (162) is bound to the execution context defined by the active order set (132) and applicable governance policy.

Upon satisfaction of admissibility by the constraint and safety grammar validator (150) and satisfaction of any required authorization evidence (162), a validation and execution interface (170) dispatches the therapeutic-device control command (142) to the therapeutic device (190). The validation and execution interface (170) enforces execution-path control by requiring a valid authorization context prior to dispatch and by receiving post-dispatch acknowledgements, negative acknowledgements, timeouts, or error indications from the therapeutic device (190). Each dispatched command is associated with a post-dispatch observation window (172).

Post-dispatch physiologic monitoring signals and therapeutic-device telemetry are evaluated by a conformance and corrective-action subsystem (180) configured to assess conformance relative to the corridor constraints (136). The conformance and corrective-action subsystem (180) applies persistence gating logic (182) such that a deviation condition (184) is asserted only when nonconformance persists for at least one of a defined persistence duration, a threshold number of consecutive samples, or a sliding time window satisfying a defined nonconformance criterion.

When a deviation condition (184) is asserted, a corrective-action controller (200) selects and initiates at least one corrective procedure via the validation and execution interface (170). The corrective procedure may include a rollback procedure restoring a prior known-safe configuration, a compensatory procedure, a device-specific safe-mode procedure, or a suspension of further autonomous therapeutic-device actuation pending clinician reauthorization. Corrective procedures are subject to admissibility validation by the constraint and safety grammar validator (150) and to authorization requirements enforced by the risk classification and authorization module (160).

Governance enforcement is applied throughout closed-loop operation by a governance and audit layer (210) configured to generate governance explanations, enforce policy constraints, and create compliant governed records. In certain embodiments, execution and completion of a therapeutic-device control command (142) are conditioned on successful creation and append of a compliant governed record to a tamper-evident audit store (212), thereby enforcing ledger-gated completion and preserving traceability of telemetry, decision rationale, authorization evidence, dispatch events, acknowledgements, outcomes, and corrective actions.

In exemplary embodiments, the system of FIG. 1 operates as a clinician-bounded closed-loop control architecture in which patient physiologic data and therapeutic-device telemetry are continuously evaluated relative to an active clinician-authored order set defining autonomy permissions and corridor constraints. When permitted, bounded therapeutic-device control commands are proposed, validated for admissibility and risk, and conditionally dispatched under authorization and governance enforcement. Post-dispatch telemetry is monitored over time to assess conformance, and sustained deviations trigger governed corrective actions or suspension of autonomous operation. Throughout operation, governance and audit mechanisms preserve traceability, authorization context, and policy compliance.

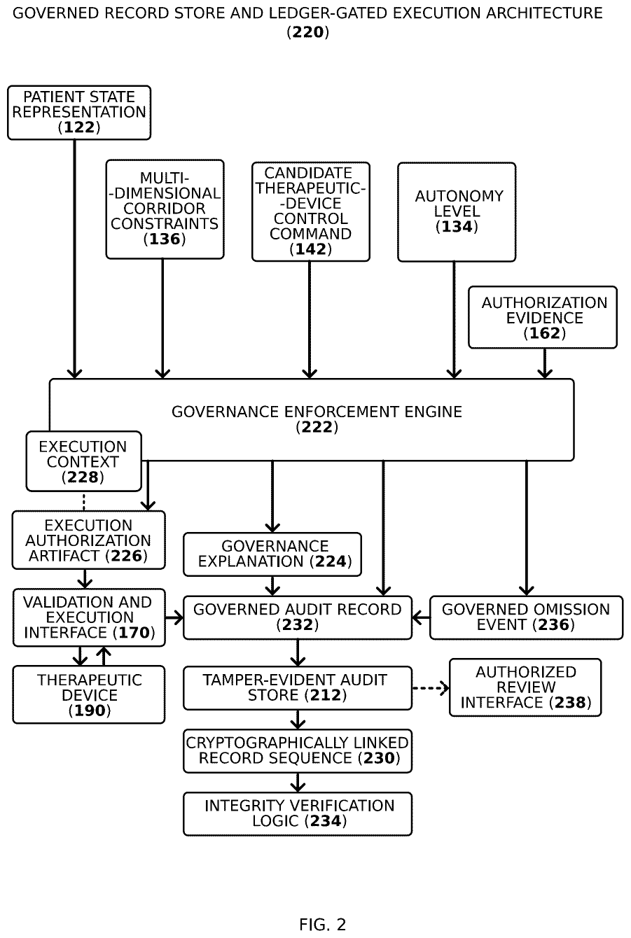

### FIG. 2

FIG. 2 illustrates an exemplary embodiment of a governed record store and ledger-gated execution architecture (220) used by the clinician-bounded closed-loop therapeutic-device control system (100) to enforce governance, auditability, and completion semantics for therapeutic-device control actions.

The governed record store architecture (220) includes a governance enforcement engine (222) configured to evaluate whether a proposed therapeutic-device control command satisfies applicable policy requirements prior to dispatch and whether execution may be treated as completed after dispatch. The governance enforcement engine (222) receives inputs including the candidate therapeutic-device control command (142), the patient state representation (122), applicable corridor constraints (136), autonomy level (134), and any required authorization evidence (162).

The governance enforcement engine (222) is configured to generate a governance explanation (224) describing the policy basis for permitting or denying execution of the candidate therapeutic-device control command (142). The governance explanation (224) identifies applicable order set identifiers, corridor identifiers, autonomy level constraints, admissibility determinations, and any missing or satisfied authorization requirements. The governance explanation (224) may be generated prior to dispatch and may be stored as part of a governed audit record.

When the candidate therapeutic-device control command (142) satisfies applicable governance constraints, the governance enforcement engine (222) issues an execution authorization artifact (226). The execution authorization artifact (226) is bound to an execution context (228) comprising one or more of a patient identifier, a therapeutic-device identifier, an action class, a corridor identifier, an order set version identifier, a permitted parameter range, and an allowable time window. The execution authorization artifact (226) is cryptographically verifiable or otherwise rendered non-forgeable and revocable at runtime.

The execution authorization artifact (226) is provided to the validation and execution interface (170), which requires a valid execution authorization artifact (226) prior to dispatching the therapeutic-device control command (142) to the therapeutic device (190). If the execution authorization artifact (226) is expired, revoked, scope-mismatched, or otherwise invalid, the validation and execution interface (170) withholds dispatch and reports a governed failure event.

The governed record store architecture (220) further includes a tamper-evident audit store (212) configured as an append-only, cryptographically linked record sequence (230). Each governed audit record (232) appended to the tamper-evident audit store (212) includes at least a reference to the candidate therapeutic-device control command (142), the associated patient state representation (122), the governance explanation (224), the execution authorization artifact (226) or a reference thereto, dispatch metadata, and any device acknowledgements received via the validation and execution interface (170).

In certain embodiments, the governance enforcement engine (222) enforces ledger-gated completion semantics by preventing the system (100) from treating a therapeutic-device control command (142) as completed unless a compliant governed audit record (232) is successfully appended to the tamper-evident audit store (212). When append fails due to storage unavailability, integrity verification failure, or policy violation, the governance enforcement engine (222) withholds completion acknowledgment and causes the system (100) to transition to a policy-defined safe behavior, including suspension of further autonomous actuation or downgrade of the autonomy level (134).

The cryptographically linked record sequence (230) comprises a sequence of audit records (232) in which each record includes a cryptographic hash or signature derived at least in part from a prior record in the sequence, thereby rendering post-hoc modification detectable. Integrity verification logic (234) periodically validates the cryptographic linkage to detect tampering or corruption.

In certain embodiments, governed audit records (232) are indexed by one or more of patient identifier, device identifier, order set version identifier, corridor identifier, autonomy level identifier, and policy identifier, thereby enabling trace reconstruction across the closed-loop lifecycle of observation, proposal, validation, authorization, dispatch, acknowledgment, outcome evaluation, and corrective action.

The governed record store architecture (220) further supports omission detection by enabling the governance enforcement engine (222) to determine whether required confirmations, reassessments, escalations, or authorization steps were not completed within required time windows. When such an omission is detected, the governance enforcement engine (222) records a governed omission event (236) in the tamper-evident audit store (212) and enforces policy-defined responses including autonomy downgrade, suspension of actuation, or escalation to clinician review.

The governed record store architecture (220) is accessible to authorized review interfaces (238) configured to retrieve governed audit records (232) and governance explanations (224) for clinical review, compliance review, or post-incident investigation. Access to governed audit records is controlled by role-based access policies and does not permit modification of stored records.

In exemplary embodiments, the architecture of FIG. 2 couples therapeutic-device actuation to governance record creation such that dispatch and/or completion of a control command is conditioned on successful generation and verification of a compliant governed audit record. Execution authorization artifacts define a bounded execution scope and are validated at dispatch time, while ledger-gated completion semantics prevent closed-loop progression when required governance records cannot be created or verified. In this manner, governance, authorization, and auditability are enforced as active control dependencies rather than post-hoc logging functions.

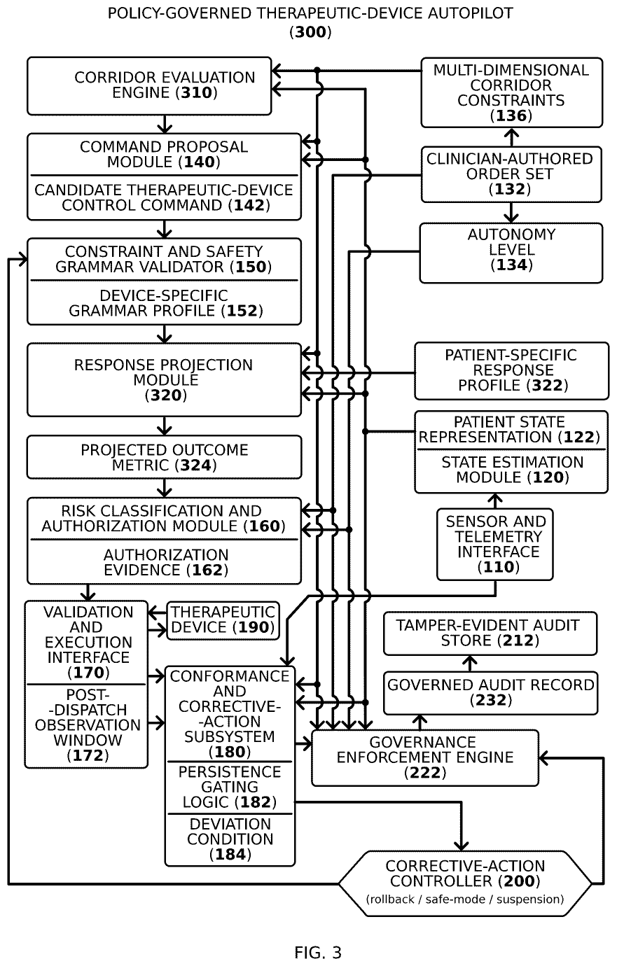

### FIG. 3

FIG. 3 illustrates an exemplary embodiment of a policy-governed therapeutic-device autopilot with physiologic corridor enforcement (300) operating as part of the clinician-bounded closed-loop therapeutic-device control system (100). The autopilot (300) is configured to continuously evaluate patient physiology relative to clinician-defined corridor constraints and to propose or execute bounded therapeutic-device control actions only within a permitted action space defined by an active order set and an autonomy level.

The policy-governed therapeutic-device autopilot (300) receives governed telemetry from the sensor and telemetry interface (110), including physiologic monitoring signals and therapeutic-device telemetry, and receives the patient state representation (122) from the state estimation module (120). The patient state representation (122) includes physiologic state variables, device status variables, temporal context, and confidence or signal-quality indicators used to control whether autonomous actuation may proceed.

The autopilot (300) includes a corridor evaluation engine (310) configured to compare one or more physiologic state variables in the patient state representation (122) against multi-dimensional corridor constraints (136) obtained from the clinician-authored order set (132). The corridor constraints (136) define target ranges, upper and lower bounds, and permissible tolerances for physiologic variables and/or therapeutic-device parameters, and may further include temporal or transition constraints that affect how quickly corrective actions may be applied.

The corridor evaluation engine (310) determines whether the patient state is within corridor, trending toward a corridor boundary, projected to violate a corridor boundary, or persistently outside a corridor boundary. The corridor evaluation engine (310) further considers confidence or signal-quality indicators included in the patient state representation (122) to determine whether available evidence is sufficient to support action or whether confirmation behavior is required.

When a deviation or projected deviation is detected and autonomous actuation is permitted by the autonomy level (134), the command proposal module (140) generates one or more candidate therapeutic-device control commands (142) intended to move the patient state toward corridor conformance. The candidate therapeutic-device control commands (142) are constrained by corridor bounds, clinician-defined step-size limits, and permitted action classes specified by the active order set (132).

Prior to dispatch, the candidate therapeutic-device control commands (142) are evaluated by the constraint and safety grammar validator (150) using the device-specific grammar profile (152) to ensure that each proposed command is admissible. The admissibility evaluation enforces semantic parameter bounds and prohibited parameter transitions, including coupled constraints between multiple therapeutic-device parameters.

The autopilot (300) further includes a response projection module (320) configured to estimate an expected physiologic and/or device response prior to dispatch of a candidate therapeutic-device control command (142). The response projection module (320) uses at least the patient state representation (122), the corridor constraints (136), and a patient-specific response profile (322) derived from prior governed therapeutic-device control actions and observed post-dispatch responses. The response projection module (320) produces one or more projected outcome metrics (324) representing expected convergence, overshoot, instability, or time-to-effect.

The projected outcome metrics (324) are provided to the risk classification and authorization module (160), which classifies each admissible candidate therapeutic-device control command (142) as low-risk or high-risk based on clinician-defined criteria specified by the clinician-authored order set (132). When a candidate therapeutic-device control command (142) is classified as high-risk, the risk classification and authorization module (160) requires authorization evidence (162) prior to dispatch.

Upon satisfaction of admissibility, risk classification, and any required authorization evidence (162), the validation and execution interface (170) dispatches the therapeutic-device control command (142) to the therapeutic device (190) and associates the dispatch with a post-dispatch observation window (172).

Following dispatch, the conformance and corrective-action subsystem (180) evaluates post-dispatch physiologic monitoring signals and therapeutic-device telemetry relative to the corridor constraints (136). The conformance and corrective-action subsystem (180) applies persistence gating logic (182) such that a deviation condition (184) is asserted only when nonconformance persists beyond defined persistence criteria, thereby preventing transient excursions from triggering corrective actions.

When a sustained deviation condition (184) is asserted, the corrective-action controller (200) selects and initiates an appropriate corrective procedure under governance control. The corrective procedure may include rollback to a prior known-safe configuration, compensatory adjustment, device-specific safe-mode transition, or suspension of further autonomous actuation pending clinician reauthorization. Corrective procedures are validated for admissibility by the constraint and safety grammar validator (150) and may require additional authorization evidence (162) depending on risk classification.

Throughout operation of the policy-governed therapeutic-device autopilot (300), governance enforcement is applied by the governance enforcement engine (222), which ensures that dispatch and completion semantics comply with policy requirements and that governed audit records (232) are created and appended to the tamper-evident audit store (212) in accordance with ledger-gated completion rules.

The sequence of evaluations and modules illustrated in FIG. 3 is exemplary, and in other embodiments one or more evaluations may be performed in parallel, reordered, repeated, or omitted under governance control while remaining within the scope of the claimed subject matter.

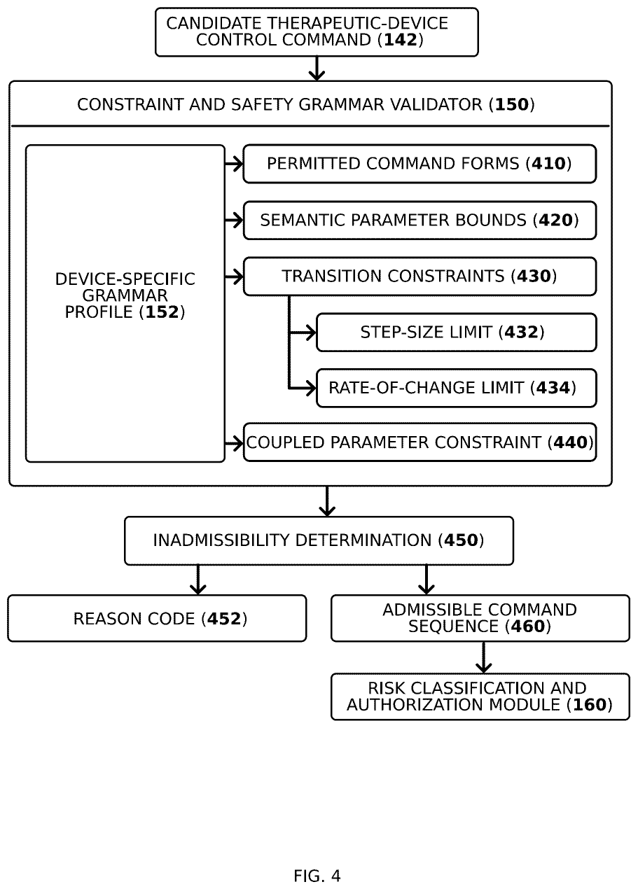

### FIG. 4

FIG. 4 illustrates an exemplary embodiment of a constraint and safety grammar validator (150) used by the clinician-bounded closed-loop therapeutic-device control system (100) to determine admissibility of candidate therapeutic-device control commands prior to dispatch to a therapeutic device (190).

The constraint and safety grammar validator (150) receives one or more candidate therapeutic-device control commands (142) generated by the command proposal module (140). Each candidate therapeutic-device control command (142) represents an intent-level adjustment to one or more therapeutic-device parameters expressed independently of device-specific command syntax.

The constraint and safety grammar validator (150) evaluates each candidate therapeutic-device control command (142) against a device-specific grammar profile (152) associated with the therapeutic device (190) to which the command would be dispatched. The device-specific grammar profile (152) defines a set of permitted command forms (410) that specify which command classes and parameter combinations are allowed for the therapeutic device (190) in a given operating mode.

The device-specific grammar profile (152) further defines semantic parameter bounds (420) specifying allowable numeric ranges for individual therapeutic-device parameters. Semantic parameter bounds (420) constrain candidate therapeutic-device control commands (142) such that parameters remain within clinically and mechanically safe limits independent of corridor constraints defined by the clinician-authored order set (132).

In addition to semantic parameter bounds (420), the device-specific grammar profile (152) defines transition constraints (430) governing how therapeutic-device parameters may change over time. Transition constraints (430) include step-size limits (432) restricting the magnitude of a parameter change between successive commands and rate-of-change limits (434) restricting how quickly a parameter may change over time.

In certain embodiments, the device-specific grammar profile (152) further defines coupled parameter constraints (440) that restrict permissible combinations of changes across two or more therapeutic-device parameters. The coupled parameter constraints (440) enforce interdependencies between parameters such that a change to one parameter may be prohibited or conditioned unless another parameter satisfies a defined relationship.

The constraint and safety grammar validator (150) applies the device-specific grammar profile (152) to evaluate each candidate therapeutic-device control command (142) for admissibility. When a candidate therapeutic-device control command (142) violates any semantic parameter bound (420), transition constraint (430), or coupled parameter constraint (440), the constraint and safety grammar validator (150) rejects the candidate therapeutic-device control command (142) and produces an inadmissibility determination (450) identifying the violated constraint.

In certain embodiments, the constraint and safety grammar validator (150) further produces a reason code (452) associated with the inadmissibility determination (450). The reason code (452) identifies the specific constraint violated and may be used by downstream governance components to generate a governance explanation (224) or to request revised authorization or corridor modification from an authorized clinician.

In some embodiments, when a candidate therapeutic-device control command (142) violates a transition constraint (430) or coupled parameter constraint (440) but could be decomposed into a sequence of admissible intermediate commands, the constraint and safety grammar validator (150) generates an admissible command sequence (460) comprising one or more intermediate commands that satisfy all applicable constraints. Each command in the admissible command sequence (460) remains subject to risk classification, authorization requirements, and governance enforcement prior to dispatch.

The admissibility determination (450) produced by the constraint and safety grammar validator (150) is provided to the risk classification and authorization module (160) and the governance enforcement engine (222). Candidate therapeutic-device control commands (142) that are rejected by the constraint and safety grammar validator (150) are not dispatched by the validation and execution interface (170) and are recorded as governed denial events in the tamper-evident audit store (212).

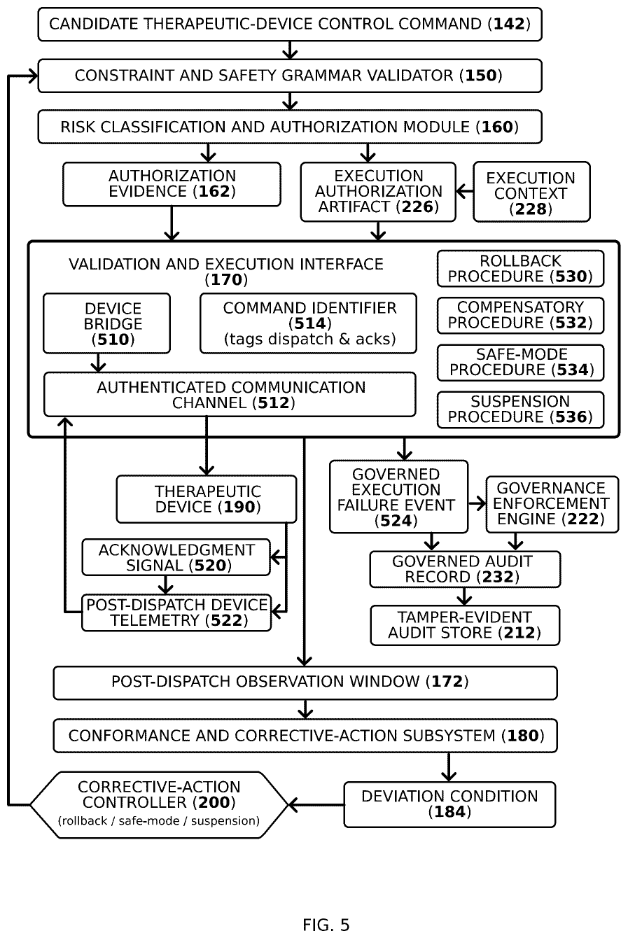

### FIG. 5

FIG. 5 illustrates an exemplary embodiment of a validation and execution interface (170) configured to enforce authorized dispatch, telemetry feedback, acknowledgment handling, and corrective-action execution for therapeutic-device control commands issued by the clinician-bounded closed-loop therapeutic-device control system (100).

The validation and execution interface (170) receives admissible candidate therapeutic-device control commands (142) that have satisfied constraint validation by the constraint and safety grammar validator (150) and any required authorization evidence (162) enforced by the risk classification and authorization module (160). Prior to dispatch, the validation and execution interface (170) verifies the presence and validity of an execution authorization artifact (226) issued by the governance enforcement engine (222) and bound to an execution context (228).

The validation and execution interface (170) includes a device bridge (510) configured to translate the candidate therapeutic-device control command (142) into a device-specific command format compatible with the therapeutic device (190). The device bridge (510) enforces scope and parameter constraints during translation to ensure that the translated command remains within the bounds validated during admissibility determination.

Upon successful translation and authorization verification, the validation and execution interface (170) dispatches the translated command to the therapeutic device (190) over a mutually authenticated communication channel (512). The validation and execution interface (170) associates each dispatch event with a command identifier (514) and a post-dispatch observation window (172) used for subsequent conformance evaluation.

The therapeutic device (190) returns execution feedback to the validation and execution interface (170) in the form of one or more acknowledgment signals (520). The acknowledgment signals (520) may include a positive acknowledgment indicating acceptance of the command, a negative acknowledgment indicating rejection of the command, a completion acknowledgment indicating successful execution, or an error indication identifying a device-side failure. The validation and execution interface (170) further detects timeouts and ambiguous completion states when expected acknowledgments are not received within defined time windows.

The validation and execution interface (170) forwards acknowledgment signals (520) and post-dispatch therapeutic-device telemetry (522) to the governance enforcement engine (222) and the conformance and corrective-action subsystem (180). When a negative acknowledgment, timeout, or ambiguous completion state is detected, the validation and execution interface (170) generates a governed execution failure event (524) and prevents further autonomous dispatch pending governance resolution.

The validation and execution interface (170) further supports execution of corrective procedures initiated by the corrective-action controller (200). Corrective procedures include rollback procedures (530) that restore one or more therapeutic-device parameters to a prior known-safe configuration, compensatory procedures (532) that apply offsetting adjustments, safe-mode procedures (534) that transition the therapeutic device (190) to a preconfigured safe mode, and suspension procedures (536) that prevent further autonomous actuation until reauthorization.

Each corrective procedure executed through the validation and execution interface (170) is subject to the same authorization verification, admissibility validation, and acknowledgment handling as an ordinary therapeutic-device control command (142). Corrective dispatch events, acknowledgments, and outcomes are recorded as governed audit records (232) in the tamper-evident audit store (212) and are associated with the deviation condition (184) that triggered the corrective action.

In certain embodiments, the validation and execution interface (170) enforces ledger-gated completion semantics by withholding completion acknowledgment of a therapeutic-device control command (142) or corrective procedure unless a compliant governed audit record (232) has been successfully appended to the tamper-evident audit store (212). When ledger append fails, the validation and execution interface (170) prevents progression of the closed-loop control sequence and transitions the system (100) to a policy-defined safe behavior.

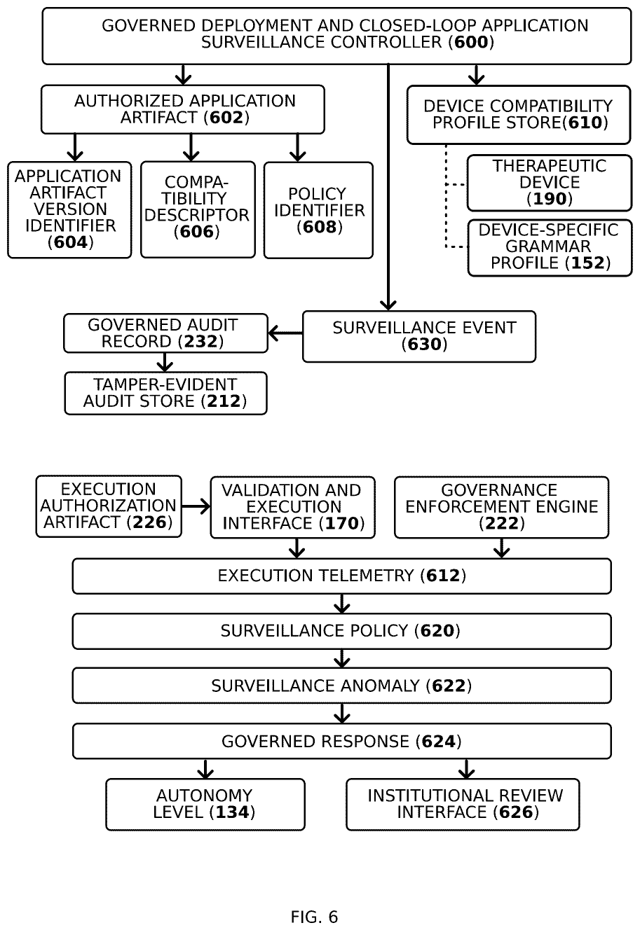

### FIG. 6

FIG. 6 illustrates an exemplary embodiment of a governed deployment and closed-loop application surveillance controller (600) configured to manage software deployment, version enforcement, and runtime surveillance for the clinician-bounded closed-loop therapeutic-device control system (100) operating in a regulated clinical environment.

The governed deployment and surveillance controller (600) is configured to maintain a registry of authorized application artifacts (602) corresponding to deployable software components that implement one or more functions of the clinician-bounded control system (100). Each authorized application artifact (602) is associated with a version identifier (604), a compatibility descriptor (606), and one or more policy identifiers (608) defining conditions under which the artifact may be executed in a clinical environment.

The governed deployment and surveillance controller (600) further maintains a device compatibility profile store (610) identifying therapeutic-device models, firmware versions, and supported command grammars (152) with which a given application artifact (602) is permitted to interact. Prior to enabling autonomous actuation, the governed deployment and surveillance controller (600) verifies that the deployed application artifact (602), the therapeutic device (190), and the applicable device-specific grammar profile (152) satisfy compatibility requirements defined by the compatibility descriptor (606).

During runtime operation, the governed deployment and surveillance controller (600) receives execution telemetry (612) from the governance enforcement engine (222) and the validation and execution interface (170). The execution telemetry (612) includes, without limitation, counts of dispatched commands, acknowledgment outcomes, authorization denials, ledger-gated completion failures, deviation conditions (184), corrective-action executions, and omission events (236).

The governed deployment and surveillance controller (600) evaluates the execution telemetry (612) relative to one or more surveillance policies (620) that define acceptable operational envelopes for autonomous or semi-autonomous behavior. Surveillance policies (620) may define thresholds for abnormal rates of command rejection, excessive rollback frequency, elevated authorization failure rates, unexpected persistence of deviation conditions (184), or other indicators of degraded or unsafe behavior.

When the governed deployment and surveillance controller (600) detects a surveillance anomaly (622) indicating that runtime behavior exceeds a defined policy threshold, the controller (600) initiates a governed response (624). The governed response (624) may include downgrading the autonomy level (134), suspending further autonomous actuation, requiring reauthorization from an authorized clinician, or triggering escalation to institutional review interfaces (626).

In certain embodiments, the governed deployment and surveillance controller (600) further enforces version-gated execution by requiring that execution authorization artifacts (226) issued by the governance enforcement engine (222) reference an approved application artifact identifier (602) and version identifier (604). When an application artifact version (604) is revoked, superseded, or placed under restricted operation, the governed deployment and surveillance controller (600) invalidates associated execution authorization artifacts (226) and prevents further dispatch through the validation and execution interface (170).

The governed deployment and surveillance controller (600) records surveillance events (630), governed responses (624), and version enforcement actions as governed audit records (232) in the tamper-evident audit store (212). Each surveillance-related audit record is associated with the applicable application artifact identifier (602), version identifier (604), policy identifier (608), and affected patient or device scope to preserve traceability.

The governed deployment and surveillance controller (600) supports staged deployment and rollback by enabling selective activation of application artifacts (602) across subsets of devices or clinical units. Execution telemetry (612) collected during staged deployment is evaluated to determine whether an application artifact (602) continues to satisfy surveillance policies (620), and failure to satisfy such policies results in automatic rollback to a prior approved application artifact version (604) under governance control.

The governed deployment and surveillance controller (600) provides institutional review interfaces (626) configured to present surveillance findings, governed responses, and version histories to authorized administrative or clinical governance personnel. Access to the institutional review interfaces (626) is restricted by role-based access controls and does not permit modification of governed audit records (232).

In exemplary embodiments, the governed deployment and surveillance controller of FIG. 6 supervises software identity, compatibility, and runtime behavior of the closed-loop system by enforcing version-gated execution and surveillance policies without introducing independent actuation authority.

1. A clinician-bounded closed-loop therapeutic-device control system for use in a regulated clinical environment, the system configured to issue and enforce therapeutic-device control commands affecting physical operation of at least one therapeutic device, comprising:
(a) a telemetry interface configured to receive real-time physiologic monitoring signals associated with a patient and therapeutic-device telemetry from the therapeutic device;
(b) a state estimation module configured to process the physiologic monitoring signals and therapeutic-device telemetry to generate a patient state representation or a functionally equivalent computed state abstraction indicative of current physiologic and device conditions;
(c) an order-set and corridor module configured to obtain, from an active clinician-authored order set, an autonomy level defining permitted classes of system action and one or more corridor constraints comprising bounded ranges, thresholds, or equivalent policy-defined limits defining permitted bounds for at least one physiologic state variable and/or therapeutic-device parameter;
(d) a command proposal module configured, when therapeutic-device actuation is permitted by the autonomy level, to generate a candidate therapeutic-device control command based on the patient state representation and the corridor constraints;
(e) a validation and execution interface configured to determine whether the candidate therapeutic-device control command is permitted under the autonomy level and corridor constraints and, when permitted, to cause dispatch of the command to the therapeutic device, directly or via an intermediary execution interface and receive post-dispatch telemetry; and
(f) a conformance and corrective-action subsystem comprising a corrective-action controller, the conformance and corrective-action subsystem being configured to evaluate post-dispatch telemetry relative to the corridor constraints over time and, upon detecting sustained deviation from the corridor constraints, to assert a deviation condition, and upon assertion of the deviation condition, to initiate, via the corrective-action controller, a corrective action affecting operation of the therapeutic device in accordance with the autonomy level.

2. The system of claim 1, further comprising
a response projection module configured, prior to dispatch of a candidate therapeutic-device control command, to estimate an expected physiologic and/or therapeutic-device response using at least the patient state representation and the corridor constraints, and
to generate a projected outcome metric indicative of a predicted response to the candidate therapeutic-device control command,
wherein the system is configured to condition dispatch of the candidate therapeutic-device control command on the projected outcome metric satisfying at least one clinician-defined risk criterion specified by the clinician-authored order set.

3. The system of claim 1, further comprising
a constraint and safety grammar validator configured to evaluate candidate therapeutic-device control commands against a device-specific grammar defining permitted command forms and prohibited parameter transitions; and
an authorization enforcement module configured to prohibit dispatch of a candidate therapeutic-device control command unless the candidate therapeutic-device control command is admissible under the device-specific grammar and satisfies an applicable authorization requirement defined by the clinician-authored order set,
wherein the candidate therapeutic-device control command is prevented from being dispatched to the therapeutic device when either the admissibility requirement or the authorization requirement is not satisfied.

4. The system of claim 3, further comprising a governance enforcement engine configured to block dispatch of a therapeutic-device control command, or to block progression of the closed-loop control system following dispatch, unless a compliant governed record is successfully created prior to such dispatch or progression, the compliant governed record describing the therapeutic-device control command, a policy basis under which the command is permitted, and any required authorization evidence, wherein blocking progression of the closed-loop control system comprises preventing the therapeutic-device control command from being treated as completed for purposes of enabling dispatch of a subsequent therapeutic-device control command.

5. The system of claim 4, wherein the governance enforcement engine generates an execution authorization artifact that defines a permitted execution scope for a therapeutic-device control command, the permitted execution scope being constrained by an execution context comprising at least one of a patient identifier, a therapeutic-device identifier, an action class, an order set version identifier, a corridor identifier, a permitted parameter range, or an allowable time window,
wherein the validation and execution interface requires the execution authorization artifact to validate that the therapeutic-device control command falls within the permitted execution scope prior to dispatch,
and wherein the execution authorization artifact is cryptographically verifiable or otherwise resistant to forgery or reuse outside the permitted execution scope at runtime.

6. The system of claim 4, wherein the compliant governed record is written to a tamper-evident audit store configured to render post-hoc modification or deletion of a recorded governed record detectable, the audit store comprising records that are logically or cryptographically linked to preserve ordering and integrity,
and wherein the system treats the therapeutic-device control command as completed for purposes of closed-loop progression only after successful creation and verification of the compliant governed record in the audit store,
and wherein failure to successfully create or verify the compliant governed record causes the system to treat the therapeutic-device control command as failed and inhibits further autonomous progression of the closed-loop control system.

7. The system of claim 1, wherein the system is configured to evaluate, prior to dispatch of a candidate therapeutic-device control command, an evidence sufficiency criterion based on at least a confidence or signal-quality indicator included in the patient state representation, and to prohibit dispatch of the candidate therapeutic-device control command when the evidence sufficiency criterion is not satisfied.

8. The system of claim 7, wherein, when the evidence sufficiency criterion is not satisfied, the system generates a governed confirmation request comprising at least one of: a request for an additional measurement, a request for increased monitoring cadence for a defined time window, or a request for clinician confirmation,
wherein generation of the governed confirmation request creates an unresolved system state that blocks subsequent autonomous therapeutic-device actuation until the confirmation request is resolved in accordance with the clinician-authored order set or applicable policy.

9. The system of claim 1, wherein the conformance and corrective-action subsystem is configured to determine whether a deviation is sustained by withholding assertion of the deviation condition in response to transient excursions and asserting the deviation condition only after nonconformance relative to the corridor constraints persists according to at least one of a defined persistence duration, a threshold number of consecutive samples, or a sliding time window satisfying a defined nonconformance criterion.

10. The system of claim 1, further comprising an omission detection module configured to automatically detect failure to perform a confirmation, reassessment, escalation, or bounded adjustment that is required by the clinician-authored order set or applicable policy within a specified time window,
and wherein, upon detecting the failure, the system records a governed omission event and enforces a control-state change by downgrading the autonomy level or suspending further autonomous therapeutic-device actuation pending clinician review or reauthorization.

11. The system of claim 4, further comprising a monitoring cadence controller configured to initiate modification of a measurement cadence of a patient monitoring device based on at least one of
(i) proximity to the corridor constraints as determined by at least one of a threshold distance to a corridor boundary, a rate of change of a monitored physiologic variable toward a corridor boundary, or a predicted corridor violation within a defined future time window, or
(ii) a deviation condition,
wherein initiation of the modification of the measurement cadence is treated as a governed action subject to the governance enforcement engine and recorded in association with the corridor constraints and the autonomy level,
and wherein the modified measurement cadence is configured to affect eligibility for dispatch of a subsequent therapeutic-device control command by causing the closed-loop control system to withhold progression unless post-dispatch telemetry received at the modified measurement cadence satisfies at least one telemetry sufficiency requirement specified by the clinician-authored order set or applicable policy, the telemetry sufficiency requirement comprising at least one of a minimum number of samples within an observation window, a minimum confidence or signal-quality score, or maintenance of the modified measurement cadence for at least a defined duration.

12. The system of claim 3, wherein the device-specific grammar profile further defines coupled transition constraints between two or more therapeutic-device parameters as pre-dispatch admissibility conditions,
and wherein the constraint and safety grammar validator is configured to reject, prior to dispatch, any candidate therapeutic-device control command that violates a coupled transition constraint and to generate a reason code identifying the violated constraint, thereby preventing execution of the rejected command.

13. The system of claim 3, wherein, upon assertion of the deviation condition, the corrective-action controller is configured to automatically select and initiate, via the validation and execution interface, at least one corrective procedure comprising a rollback procedure restoring the therapeutic device to a prior known-safe configuration or a device-specific safe-mode procedure transitioning the therapeutic device to a preconfigured safe configuration,
and wherein initiation and execution of the corrective procedure are subject to admissibility validation by the constraint and safety grammar validator and to satisfaction of any required authorization evidence, such that the corrective procedure is executed through the same governed execution path as therapeutic-device control commands.

14. The system of claim 1, wherein the conformance and corrective-action subsystem is further configured to automatically suspend autonomous therapeutic-device actuation and transition the system to a report-only or clinician-review mode when a prerequisite for autonomous actuation is not satisfied,
wherein the prerequisite comprises at least one of insufficient evidence quality, conflicting telemetry, failure to receive a required authorization, failure to validate a candidate therapeutic-device control command, or inability to confirm completion of a previously dispatched therapeutic-device control command.

15. A computer-implemented method for clinician-bounded closed-loop control of a therapeutic device in a regulated clinical environment, the method comprising:
(a) receiving, via a telemetry interface, physiologic monitoring signals associated with a patient and therapeutic-device telemetry from a therapeutic device;
(b) processing the physiologic monitoring signals and therapeutic-device telemetry to generate a patient state representation indicative of current physiologic and device conditions;
(c) obtaining, from an active clinician-authored order set, an autonomy level defining permitted classes of system action and one or more corridor constraints defining permitted bounds for at least one physiologic state variable and/or therapeutic-device parameter;
(d) when therapeutic-device actuation is permitted by the autonomy level, generating a candidate therapeutic-device control command based on the patient state representation and the corridor constraints;
(e) determining whether the candidate therapeutic-device control command is permitted under the autonomy level and the corridor constraints;
(f) when permitted, causing dispatch of the candidate therapeutic-device control command to the therapeutic device;
(g) receiving post-dispatch therapeutic-device telemetry; and
(h) evaluating the post-dispatch telemetry relative to the corridor constraints over time and, upon detecting sustained deviation from the corridor constraints, initiating a corrective action affecting operation of the therapeutic device.

16. The method of claim 15, further comprising, prior to dispatching the candidate therapeutic-device control command, estimating an expected physiologic or device response to the candidate therapeutic-device control command using the patient state representation and the corridor constraints to generate a projected outcome metric, wherein dispatching the candidate therapeutic-device control command is conditioned on the projected outcome metric satisfying at least one clinician-defined risk criterion specified by the clinician-authored order set.

17. The method of claim 15, further comprising evaluating the candidate therapeutic-device control command against a device-specific safety grammar defining permitted command forms and prohibited parameter transitions, and prohibiting dispatch of the candidate therapeutic-device control command unless the candidate therapeutic-device control command is admissible under the safety grammar and satisfies an applicable authorization requirement defined by the clinician-authored order set.

18. A non-transitory computer-readable medium storing instructions that, when executed by one or more processors, cause performance of operations for clinician-bounded closed-loop control of a therapeutic device in a regulated clinical environment, the operations comprising:
(a) receiving physiologic monitoring signals associated with a patient and therapeutic-device telemetry from a therapeutic device;
(b) generating a patient state representation indicative of current physiologic and device conditions;
(c) obtaining, from an active clinician-authored order set, an autonomy level defining permitted classes of system action and one or more corridor constraints defining permitted bounds for at least one physiologic state variable and/or therapeutic-device parameter;
(d) generating a candidate therapeutic-device control command when therapeutic-device actuation is permitted by the autonomy level;
(e) determining whether the candidate therapeutic-device control command is permitted under the autonomy level and the corridor constraints;
(f) dispatching the candidate therapeutic-device control command to the therapeutic device when permitted;
(g) receiving post-dispatch therapeutic-device telemetry; and
(h) initiating a corrective action affecting operation of the therapeutic device upon detecting sustained deviation from the corridor constraints over time.

19. The non-transitory computer-readable medium of claim 18, wherein the operations further comprise generating a projected outcome metric indicative of an expected physiologic or device response to the candidate therapeutic-device control command, and conditioning dispatch of the candidate therapeutic-device control command on the projected outcome metric satisfying a clinician-defined risk criterion.

20. The non-transitory computer-readable medium of claim 18, wherein the operations further comprise evaluating the candidate therapeutic-device control command against a device-specific safety grammar defining permitted command forms and prohibited parameter transitions, and preventing dispatch of the candidate therapeutic-device control command unless the candidate therapeutic-device control command is admissible under the safety grammar and satisfies an authorization requirement defined by the clinician-authored order set.
